(19) United States
(12) Patent Application Publication
(10) Pub. No.: US 2003/0138778 A1
(43) Pub. Date: Jul. 24, 2003

(54) Prediction of disease-causing alleles from sequence context

(76) Inventors: Harold R. Garner, Coppell, TX (US); Monica M. Horvath, Dallas, TX (US); John W. Fondon III, Lewisville, TX (US); Alexander Pertsemlidis, Coppell, TX (US)

Correspondence Address: Sanford E. Warren, Gardere Wynne Sewell LLP, Suite 3000, 1601 Elm Street, Dallas, TX, 75201-4761, US

(21) Appl. No.: 09/998,904

(22) Filed: Nov. 30, 2001

(51) Int. Cl. C12Q 1/68; G06G 7/48; G06G 7/58; G06F 19/00; G01N 33/48; G01N 33/50

(52) U.S. Cl. 435/6; 702/20; 703/11

(57) ABSTRACT

An apparatus, system and method for predicting single nucleotide polymorphisms (SNPs) is disclosed. The present invention generally includes steps for obtaining a variation predictiveness matrix and predicting one or more single nucleotide variations of a nucleic acid sequence based on the variation predictiveness matrix. The variation predictiveness matrix may be made by calculating the variation frequency from a first base to a second base in a dataset of two or more bases and determining a variation predictiveness value from the calculated variation frequency.

### TECHNICAL FIELD OF THE INVENTION

The present invention relates in general to the field of genetic testing, and more particularly, to an apparatus, method and system for predicting single nucleotide polymorphisms.

### BACKGROUND OF THE INVENTION

Without limiting the scope of the invention, its background is described in connection with the identification of single nucleotide polymorphisms, as an example.

Since the completion of a draft human genome sequence, post-genomic science has had the information to empower whole organism-driven research that complements current technique-driven and molecule-driven methods. For example, the interaction of many proteins may be studied on an organ level to elucidate complex problems such as cell-cell signaling and its relation to disease. A number of projects have attempted to catalogue disease-causing DNA variations, a goal that would revolutionize common practices of modern medicine but is based on DNA sequencing. Sequencing is the historical method of discovering alleles related to Mendelian diseases, which have been amongst the first disease variants discovered. True post-genomic approaches represent a new way of thinking about science where the best start for a new experiment is often a computational approach. Large web-based databases exist for a wide range of experimental data that, when analyzed, may provide invaluable knowledge that can increase the chance of in-house experimental success.

Some studies have tried to correlate disease susceptibility to the most common class of variation, single nucleotide polymorphisms (SNPs). SNPs are germ line point mutations that occur at a frequency of >1% in the global human population, although there is poor adherence to this definition within the SNP community. Often an ethnically or disease-stratified population (<100 individuals) is genotyped and any point variation discovered within that small group is described as a SNP. Using straight sequencing the probability of discovering a polymorphic allele is dependent on amassing the correct population stratification. For example, the frequencies of SNPs discovered in the BRCA1 gene from a group of several hundred individuals diagnosed with advanced breast cancer inaccurately portrays the global variation of the gene. The inaccuracy is because mutations discovered with allele frequencies of >1% in that focused group of people will be championed as a SNP and form the bulk of many a candidate disease gene/allele association study.

Numerous SNP-hunting projects have emerged to link single base variation to disease using DNA sequencing, such as Celera's SNP database, the SNP Consortium, and work done by the National Human Genome Research Institute. Discovered mutations may relate to disease susceptibility either through direct association, where the allele has a deleterious effect on fitness and will be found at a higher frequency in a disease population verses an unaffected population, or indirect association, where the variant is a member of a set of alleles in linkage disequilibrium with another allele known to be causative of disease. The indirect association method relies on the hypothesis that each allele must have arisen concomitantly in a particular individual at some time in the past causing the profile of linked polymorphisms in the altered region to be inherited along with the disease-causing allele. The classification of newly discovered point mutations is not immediately apparent. Furthermore, the problem with nearly all large-scale variation searching is that genotyping practices limit finding to discovering only very common alleles. Only a handful of individuals (˜24) are screened due to the time and expense associated with DNA sequencing, which often misses even those variants with frequencies in the 1-5% range. The difficulty in screening is compounded by the fact that the sequencing error rate is often higher than the allele frequency causing many false positives.

### SUMMARY OF THE INVENTION

Gene mutation contributes to virtually every medical human affliction, and much of the biotechnology industry is devoted to making an association between a gene and a disease condition to improve diagnosis, treatment and disease prevention. The completion of the human genome sequencing project has opened opportunities for all types of variation studies, especially those of single nucleotide polymorphism (SNP), which are single base positions in genes that may display multiple alleles. The nature, frequency and location of gene lesions causing human genetic disease are non-random and determined in part by the local DNA sequence environment. As used herein a SNP is a variant or point mutation.

Once a given mutation has arisen, the likelihood that it will receive clinical attention depends on the level of effects that the mutation may have on protein structure and function. Currently, studies on large numbers of missense and nonsense mutations in a specific gene are rare because these mutations are extremely difficult to pinpoint. What is currently unavailable is a system and method for recognizing the non-random nature of gene lesions and to distinguish as well as predict the occurrence of nonsynonymous (amino acid altering) point mutations. The ability to predict mutations based on the non-random nature of gene lesions would allow for the identification of candidate “hotspots” in the genome; disease-specific DNA variations that should be genotyped when any individual is screened for any disease. Generating fast, accurate and predictive mutations for disease-linked gene lesions removes the limitations of time and cost associated with the methods available currently and permits large scale genotyping for all affected or non-affected persons.

The apparatus, system and method of the present invention makes is possible to predict likely point mutations from a wild-type DNA sequence context at a rate usefully better than random. Here, the invention considers two major categories of DNA point mutations that occur in the coding region of a gene: (i) point substitutions that alter the composition of the encoded protein as to effect a phenotype, and (ii) neutral variations (or substitutions) that may not alter protein structure either because the substitution is synonymous or accepted by the protein. Naturally, the first type of DNA point mutation would be represented by studies seeking to pinpoint one or more mutations that cause a disease and therefore rare in the natural population due to selective pressures. Given that neutral substitutions would not be subject to such constraints, it is expected that these variations are quite common, easy to locate, yet may be pharmacologically irrelevant.

The present inventors have pioneered a novel statistical analysis tool, developed to predict point mutations, known as SNIDE (Single Nucleotide variation IDEntification). The tool is based on the statistical analysis of DNA variation patterns and uses that statistical analysis to identify disease-causing mutations. With the present invention it is now possible to predict likely phenogenic point mutations, herein known as pSNPs, from sequence context. This invention provides an improved set of targets for exhaustive genotyping of one or many individuals with a known or unknown disorder. It is important to note that the present invention may be used for persons known to harbor even the most complex of diseases caused by a combination of mutations in numerous genes.

SNIDE allows the user to identify one or more point mutations in a set of genes thought to be associated, with, e.g., cardiac disease or other multi-gene disorders, and to genotype a large panel of individuals with the disorder. The present invention includes computationally validated data for predicting pSNPs even when only wild-type nucleic acid sequence information is available for a given gene. The predictiveness of SNIDE has been verified in two ways: (i) by testing subsets of observed SNPs in the mutation database with SNIDE predictions (i.e., performed with software that analyzed the p53 and CFTR genes by removing them from the “training” database or HGMD and then checking SNIDE analysis of the genes against the observed SNPs; here, agreement was correlated); and (ii) by DNA sequencing of regions of candidate genes predicted to be of high mutation ranking in an affected population and comparing the findings with the SNIDE prediction. Finally, SNIDE may also incorporate information about the family of the encoded protein and test the predictions in a disease population.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is a graph that compares the probability of detecting an allele of known frequency in a given population/drawing/flow diagram/illustrative cross section;

FIGS. 2A-2C show the distribution of nonsynonymous codon mutation classes in: (2a) the whole HGMD; (2b) the CFTR gene; and (2c) the Factor IX gene;

FIG. 3 is a graph that demonstrates the computational validation of SNIDE point mutation predictions; and

FIG. 4 is a DNA sequence chromatogram that shows the mutation (THR→MET) at or about position 875.

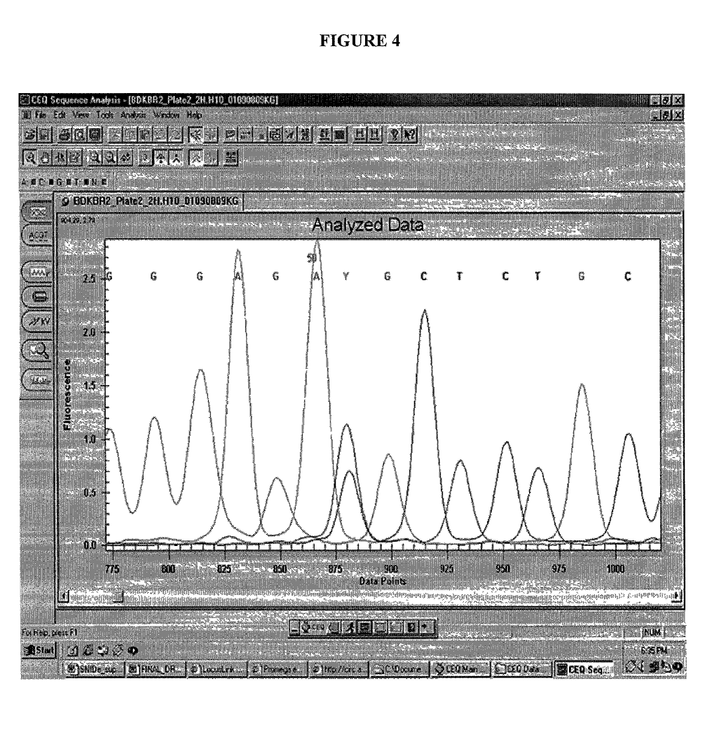

FIG. 5 is a flowchart describing the construction and deployment of SNIDE.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

All technical and scientific terms used herein have the same meaning as commonly understood by one of ordinary skill in the art to which this invention belongs, unless defined otherwise. Methods and materials similar or equivalent to those described herein may be used in the practice or testing of the present invention, the generally used methods and materials are now described. All publications mentioned herein are incorporated herein by reference to disclose and describe the methods and/or materials in connection with which the publications are cited.

As used throughout the present specification the following abbreviations and symbols are used: SNIDE, Single Nucleotide variation IDEntification; SNooP, single nucleotide polymorphism; SNP, single nucleotide polymorphism; pSNP, phenogenic point mutation; nSNP, neutral point mutation; MS, mass spectroscopy; HGMD, Human Genome Mutation Database; MALDI, matrix assisted laser desorption ionization; MALDI-TOF MS, matrix assisted laser desorption ionization time-of-flight mass spectroscopy; ζ, predictiveness value.

As used herein “nucleic acid” is either DNA, RNA, single-stranded or double-stranded and any chemical modifications thereof, including both natural and artificial modifications, protein nucleic acids or even locked nucleic acids. Modifications include, but are not limited to, those that provide other chemical groups that incorporate additional charge, polarizability, hydrogen bonding, electrostatic interaction, and fluxionality to the individual nucleic acid bases or to the nucleic acid as a whole.

A “nucleic acid target element” is a determinable sequence that contains at least one peptide located at a different location on the substrate. The determinable sequence comprises either DNA, RNA, single-stranded or double-stranded and any chemical modifications thereof. Modifications include, but are not limited to, those that provide other chemical groups that incorporate additional charge, polarizability, hydrogen bonding, electrostatic interaction, and fluxionality to the individual nucleic acid bases or to the nucleic acid as a whole. The determinable sequence can further be portions of structural, metabolic, transcriptional or other genes, including ones that code for a proteases, receptors, channels, synaptic proteins, cell-cell or cell-matrix interactions, immune or inflammatory responses, cell signaling, molecular chaperones or other carrier proteins, molecular synthesis, cell cycle regulation, cell growth, cell proliferation, or cell death.

As defined herein, a “wild type” sequence, whether found in a coding, non-coding or interface sequence is an allelic form of sequence that performs the natural or normal function for that sequence. Therefore, as used herein a wild type sequence includes multiple allelic forms of a cognate sequence, for example, multiple alleles of a wild type sequence may encode silent or conservative changes to the protein sequence that a coding sequence encodes. A “mutant” sequence is defined herein as one in which at least a portion of the functionality of the sequence has been lost, for example, changes to the sequence in a promoter or enhancer region will affect at least partially the expression of a coding sequence in an organism. A “mutation” in a sequence as used herein is any change in a nucleic acid sequence that may arise such as from a deletion, addition, substitution, or rearrangement. The mutation may also affect one or more steps that the sequence is involved in. For example, a change in a DNA sequence may lead to the synthesis of an altered protein, one that is inactive, or to an inability to produce the protein. A “mutation frequency” as used herein is the frequency or rate with which a particular mutation appears in a particular dataset. Mutation frequency may also be the frequency at which any mutation appears in the whole dataset.

The term “variation” variation is used throughout the specification as a difference in nucleic acid or protein sequence. A variation includes both conservative (or synonymous) changes to a sequence or non-conservative (nonsynonymous) changes to the underlying sequence. The variations may occur at a specific locus, e.g., a SNP that may be found in one or more sequences, in a vector, plasmid, phage, bacterium, fungi, prokaryotic or eukaryotic cell, among individuals, groups, or populations. A “variation frequency” as used herein is the frequency or rate with which a particular variation appears in a particular dataset. Variation frequency may also be the frequency at which any variation appears in the whole dataset.

A “variation predictiveness matrix” is defined herein as a table, list or mathematical matrix generated from empirical sequence data that describes the expectation of every possible base to base mutation class to occur in one or more sequences as calculated from that base usage and frequency in a mutation database. The variation predictiveness matrix is capable of quantifying and qualifying, independently or concurrently, the likelihood or frequency of a sequence change occurring in a given nucleic acid sequence and/or the likelihood or frequency that the sequence change will have an effect on function, for example, on gene expression, exon expression, translocations, conservative and non-conservative amino acid changes, transcription, translation, termination, secondary, tertiary or quaternary DNA, RNA or protein structure, protein-protein interactions, biochemical activity, cell transport, signal transduction, intra and extracellular messengers, methylation, shuffling, clustering, splicing, message stability, protein stability, post-translational modifications, and the like. The variation predictiveness matrix is generally a list, chart, table or matrix that contains a predictiveness value, ζ, that may include, e.g., the likelihood or frequency of a sequence or polymorphism change occurring in a given nucleic acid base in a sequence and/or the likelihood or frequency that the sequence or polymorphism change will have an effect on function. The predictiveness value may also incorporate other factors that affect the overall score, value or number assigned for the specific matrix. Furthermore, the user of the matrix may change the threshold value of the score assigned to a base using the predictiveness value to increase the accuracy of scan or determination of the likelihood that a change in the sequence, polymorphism or mutation will have an effect at a later stage, e.g., a nonsynonymous change in protein sequence.

In one example of a variation predictiveness matrix, the variation may occur in codon usage that causes a nonsynonymous mutation that is likely to occur and that has a physiological effect. In this case the matrix is a “codon polymorphism predictiveness matrix,” in which the mutation from a first codon to a distinct second codon at the same location has a measurable effect. Measurable effect as used herein may include, for example, changes in gene expression, exon usage or expression, translocations, conservative and non-conservative amino acid changes, transcription, translation, termination, secondary, tertiary or quaternary DNA, RNA or protein structure, protein-protein interactions, biochemical or electrical activity, cell transport, signal transduction, intra and extracellular messengers, methylation, shuffling, clustering, splicing, message stability, protein stability, post-translational modifications, and the like.

The variation predictiveness matrix will often be normalized. The term “normalized” as used herein is to scale numerical data so that it can be referenced against a chosen standard value, for example, the variation predictiveness matrix may be normalized for the codon usage of a particular target organism. Codon usage tables are well known to those of skill in the art and are incorporated herein by reference.

The terms “a sequence essentially as set forth in SEQ ID NO. (#)”, “a sequence similar to”, “nucleotide sequence” and similar terms, with respect to nucleotides, refers to sequences that substantially correspond to any portion of the sequence identified herein as SEQ ID NO,: 1. These terms refer to synthetic as well as naturally-derived molecules and includes sequences that possess biologically, immunologically, experimentally, or otherwise functionally equivalent activity, for instance with respect to hybridization by nucleic acid segments, or the ability to encode all or portions of gene or genomic sequence activity. Naturally, these terms are meant to include information in such a sequence as specified by its linear order.

The term “homology” refers to the extent to which two nucleic acids are complementary. There may be partial or complete homology. A partially complementary sequence is one that at least partially inhibits a completely complementary sequence from hybridizing to a target nucleic acid and is referred to using the functional term “substantially homologous.” The degree or extent of hybridization may be examined using a hybridization or other assay (such as a competitive PCR assay) and is meant, as will be known to those of skill in the art, to include specific interaction even at low stringency.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “sequences” as used herein is used to refer to nucleotides or amino acids, whether natural or articifical, e.g., modified nucleic acids or amino acids. When describing “transcribed nucleic acids” those sequence regions located adjacent to the coding region on both the 5′, and 3′, ends such that the deoxyribonucleotide sequence corresponds to the length of the full-length mRNA for the protein as included. The term “gene” encompasses both cDNA and genomic forms of a gene. A gene may produce multiple RNA species that are generated by differential splicing of the primary RNA transcript.

The term “altered”, or “alterations” or “modified” with reference to nucleic acid or polypeptide sequences is meant to include changes such as insertions, deletions, substitutions, fusions with related or unrelated sequences, such as might occur by the hand of man, or those that may occur naturally such as polymorphisms, alleles and other structural types, e.g., chimeric sequences. Alterations encompass genomic DNA and RNA sequences that may differ with respect to their hybridization properties using a given hybridization probe. Alterations of polynucleotide sequences for a target sequence, or fragments thereof, include those that increase, decrease, or have no effect on functionality. Alterations of polypeptides refer to those that have been changed by recombinant DNA engineering, chemical, or biochemical modifications, such as amino acid derivatives or conjugates, or post-translational modifications.

The term “control sequences” refers to DNA or RNA sequences necessary for the expression of an operably linked coding sequence in a particular host organism. The control sequences that are suitable for prokaryotes, for example, include a promoter, optionally an operator sequence, a ribosome binding site, and transcriptional terminators.

As used herein the terms “protein”, “polypeptide” or “peptide” refer to compounds comprising amino acids joined via peptide bonds and are used interchangeably, whether modified or not.

As used herein, the term “endogenous” refers to a substance the source of which is from within a cell. Endogenous substances are produced by the metabolic activity of a cell. Endogenous substances, however, may nevertheless be produced as a result of manipulation of cellular metabolism to, for example, make the cell express the gene encoding the substance.

As used herein, the term “exogenous” refers to a substance the source of which is external to a cell. An exogenous substance may nevertheless be internalized by a cell by any one of a variety of metabolic or induced means known to those skilled in the art.

A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed, excised or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand.

The term “gene of interest” as used here refers to a gene, the function and/or expression of which is desired to be investigated, or the expression of which is desired to be regulated, by the present invention. The present invention may be useful in regard to any gene of any organism, whether of a prokaryotic or eukaryotic organism.

The term “hybridize” as used herein, refers to any process by which a strand of nucleic acid binds with a complementary strand through base pairing. Hybridization and the strength of hybridization (i.e., the strength of the association between the nucleic acid strands) is impacted by such factors as the degree of complementary between the nucleic acids, stringency of the conditions involved, the melting temperature of the formed hybrid, and the G:C (or U:C for RNA) ratio within the nucleic acids.

The terms “complementary” or “complementarity” as used herein, refer to the natural binding of polynucleotides under permissive salt and temperature conditions by base-pairing. For example, for the sequence “A-G-T” binds to the complementary sequence “T-C-A”. Complementarity between two single-stranded molecules may be partial, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands.

The term “homology,” as used herein, refers to a degree of complementarity. There may be partial homology or complete homology (i.e., identity). A partially complementary sequence is one that at least partially inhibits an identical sequence from hybridizing to a target nucleic acid; it is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence. When used in reference to a single-stranded nucleic acid sequence, the term “substantially homologous” refers to any probe which can hybridize (i.e., it is the complement of) the single-stranded nucleic acid sequence under conditions of low stringency as described. As known in the art, numerous equivalent conditions may be employed to comprise either low or high stringency conditions. Factors such as the length and nature (DNA, RNA, base composition) of the sequence, nature of the target (DNA, RNA, base composition, presence in solution or immobilization, etc.), and the concentration of the salts and other components (e.g., the presence or absence of formamide, dextran sulfate and/or polyethylene glycol) are considered and the hybridization solution may be varied to generate conditions of either low or high stringency different from, but equivalent to, the above listed conditions.

The term “antisense,” as used herein, refers to nucleotide sequences that are complementary to a specific DNA or RNA sequence. The term “antisense strand” is used in reference to a nucleic acid strand that is complementary to tile “sense” strand. Antisense molecules may be produced by any method, including synthesis by ligating the gene(s) of interest in a reverse orientation to a viral promoter which permits the synthesis of a complementary strand. Once introduced into a cell, the transcribed strand combines with natural sequences produced by the cell to form duplexes. These duplexes then block either the further transcription or translation. In this manner, mutant phenotypes may also be generated. The designation “negative” is sometimes used in reference to the antisense strand, and “positive” is sometimes used in reference to the sense strand. The term also is used in reference to RNA sequences that are complementary to a specific RNA sequence (e.g., mRNA). Included within this definition are antisense RNA (“asRNA” ) molecules involved in genetic regulation by bacteria. Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation. (−) (i.e., “negative”) is sometimes used in reference to the antisense strand with the designation (+) sometimes used in reference to the sense (i.e., “positive”) strand.

As used herein, the term “selectable marker” refers to the use of a gene that encodes an enzymatic activity and which confers the ability to grow in medium lacking what would otherwise be an essential nutrient (e.g., the HIS3 gene in yeast cells); in addition, a selectable marker may confer resistance to an antibiotic or drug upon the cell in which the selectable marker is expressed. A review of the use of selectable markers in mammalian cell lines is provided in Sambrook, J. et. al., Molecular Cloning: A Laboratory Manual, 2nd ed., Cold Spring Harbor Laboratory Press, New York (1989) pp.16.9-16.15.

As used herein, the term “vector” is used in reference to nucleic acid molecules that transfer DNA segment(s) from one cell to another. The term “vehicle” is sometimes used interchangeably with “vector.” The term “vector” as used herein also includes expression vectors in reference to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “amplify”, when used in reference to nucleic acids refers to the production of a large number of copies of a nucleic acid sequence by any method known in the art. Amplification is a special case of nucleic acid replication involving template specificity. Template specificity is frequently described in terms of “target” specificity. Target sequences are “targets” in the sense that they are sought to be sorted out from other nucleic acid. Amplification techniques have been designed primarily for this sorting out.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic 'd strand is induced, (i.e., in the presence of nucleotides and an inducing agent such as DNA polymerase and at a suitable temperature and pH). The primer may be single stranded for maximum efficiency in amplification but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g. ELISA, as well as enzyme-based histochemical assays), fluorescent, radioactive, and luminescent systems. It is not intended that the present invention be limited to any particular detection system or label.

As used herein, the term “target” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

The word “specific” as commonly used in the art has two somewhat different meanings. The practice is followed herein. “Specific” refers generally to the origin of a nucleic acid sequence or to the pattern with which it will hybridize to a genome, e.g., as part of a staining reagent. For example, isolation and cloning of DNA from a specified chromosome results in a “chromosome-specific library”. Shared sequences are not chromosome-specific to the chromosome from which they were derived in their hybridization properties since they will bind to more than the chromosome of origin. A sequence is “locus specific” if it binds only to the desired portion of a genome. Such sequences include single-copy sequences contained in the target or repetitive sequences, in which the copies are contained predominantly in the selected sequence.

There are two competing models describing allelic diversity: the common-disease common-variant hypothesis and the multi-equivalent risk model. The common-disease common-variant hypothesis proposes that there is a small pool of common polymorphic disease alleles that cause common diseases. Those depending on these models rely on the idea that common allelic variants account for a substantial portion of the population risk in a usefully predictive way. A crippling fallacy with this model is that phenotypic frequency does not necessarily estimate the genetic risk if the common disease in question is also heavily influenced by environmental factors. For example, cardiac disease, the leading cause of death in the United States, has been estimated to have a maximum heritability of 34% in whites and 53% in blacks. In addition, a correlation of cardiac disease incidence with spouses has been found. Smoking, obesity, and physical inactivity are just examples of environmental factors that are known to play a considerable role in disease risk even in the absence of a genetic component. Therefore, it does not follow necessarily that a common disease should be influenced by comparably common alleles alone. Another problem with the common-disease common-variant hypothesis is that so-called “common” diseases are often not a single disease but composed of multiple disorders displaying similar phenotypes, e.g., long QT syndrome, cardiomyopathy, and atherosclerosis are often described as cardiac diseases but each remain distinct and are, themselves, caused by one or more mutations in separate genes.

The competing model of allelic diversity underlying disease susceptibility is the multi-equivalent risk model. This model assumes that for any disease there is a large pool of risk alleles each having very low population frequency; the cumulative frequency of the risk alleles may be considerable, but the exact frequency of any one allele is low. This assumption complements the theory of natural selection because point mutations having a marked effect on phenotype, such as nonconservative mutations in the coding regions of genes, would be expected to have low population frequencies. In fact, a mutation-discovery approach biased against rare variants misses the very alleles that are likely to be functionally important. The present invention, SNIDE, is designed to seek mutations that exist under this model.

The present invention may even be used to analyze the likelihood of occurrence and effect of epigenetic events. Methylation of nucleic acids is an example of an epigenetic event that occurs and that has effects on, e.g., transcription. Methylation of cytosines in CpG dinucleotides is an important mechanism of transcriptional regulation. Methylation is involved in a variety of normal biological processes such as X chromosome inactivation and transcriptional regulation of imprinted genes. Aberrant methylation of cytosines can also effect transcriptional inactivation of certain tumor suppressor genes, associated with a number of human cancers. Cytosine methylation in CpG-rich areas (CpG islands) located in the promoter regions of some genes is of special regulatory importance. Therefore, wide scope mapping of methylation sites in CpG islands is important for understanding both normal and pathological cellular processes. Furthermore, methylation of certain sites may serve as an important marker for early diagnosis and treatment decisions of some cancers. Methylation site databases may be used to obtain sequences for comparison using the present invention to predict SNPs in sequences that are likely to cause or delete a methylation site that has the effect of increasing or reducing gene transcription.

A variety of methods have been used to identify sites of DNA methylation. One common method has relied on the inability of restriction endonucleases to cleave sequences that contain one or more methylated cytosines. Genomic DNA is fragmented with appropriate restriction enzymes and cleavage at the site of interest is probed electrophoretically or by PCR. This method provides an analysis of some potential methylation sites, but it is limited to sites that fall within the recognition sequences of methylation-sensitive restriction enzymes. Other methods rely on the differential chemical reactivities of cytosine and 5-methyl cytosine with reagents such as sodium bisulfite, hydrazine, or permanganate. In the case of hydrazine and permanganate, differential strand cleavage between methylated and unmethylated cytosines is examined in a similar fashion to that used when cleavage is done with restriction enzymes.

Treatment with sodium bisulfite may also be used to convert methylated and unmethylated DNA to different sequences. Under appropriate conditions, unmethylated cytosines in DNA react with sodium bisulfite to yield deoxyuridine, which behaves as thymidine in Watson-Crick hybridization and enzymatic template-directed polymerization. Methylated cytosines, however, are unreactive, and behave as cytosine in Watson-Crick hybridization and enzymatic template-directed polymerization. Sequence differences resulting from bisulfite treatment can be assessed in any of several ways. One way is with standard sequencing by primer extension (Sanger sequencing). This method has the disadvantage of limited throughput. Another way to identify sites, termed methylation-specific PCR, uses a set of PCR primers specific to the sequences resulting from bisulfite treatment of either methylation state at a given site. Effective amplification using one primer from the set indicates methylation, whereas effective amplification using the other primer indicates unmethylated cytosine at the site being amplified. This method has the disadvantage of low sample throughput in addition to the disadvantage that only one potential site of methylation is probed in an assay.

Multiple CpG dinucleotides of unknown methylation state will often be sufficiently proximal to each other in sequences to be analyzed that the probe will include one or more CpG dinucleotides in addition to the central one being analyzed. If a methylation state is assumed for these additional sites in the design of the probe sequence, the probe affinity for the analyte will be diminished whenever the assumed methylation state is not the actual methylation state. Including on the array additional probes that accommodate all possible methylation states may compensate for the resulting decrease in signal.

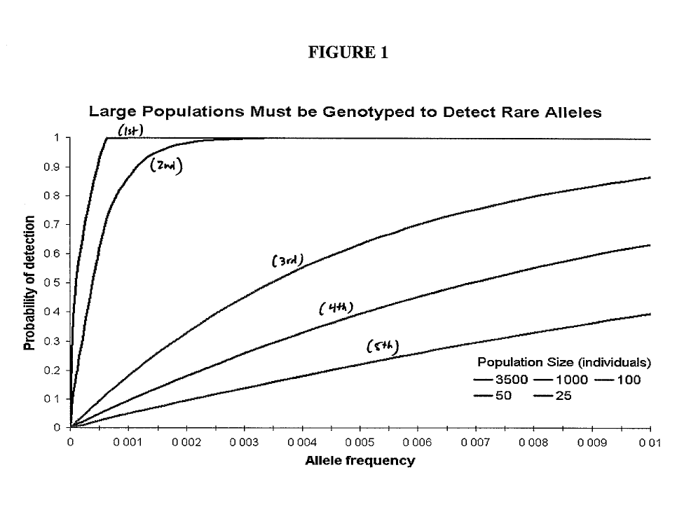

FIG. 1 demonstrates mathematically the reduction in scope caused by genotyping only small sample sizes by comparing the probability of detecting an allele of known frequency in a given population (for population size curves from left to right: 1^{st} curve, 3500; 2^{nd} curve 1000; 3^{rd} curve, 100; 4^{th} curve, 50; 5^{th} curve, 25). The probability of detection is calculated as P=1−(1−X)^{2Y} where X is the allele frequency and Y is the population size. Rare alleles (frequency <1%) are unlikely to be discovered in populations smaller than 50 individuals. A population of 3500 is sufficient (97% chance) to detect alleles having frequencies as low as 0.0005. On the other hand, there is only a 64% chance of discovering an allele of frequency 1% using a population of 50 individuals. This of course means that for all alleles of even lower frequency, a geneticist will more often miss than discover them in that 50-person population.

Clearly, the multi-equivalent risk and common-disease common-variant models represent two largely divergent models. To maximize chances of success in disease mapping, it is critical that the analytical approach is able to detect subtle genetic effects under a variety of genetic models. Current variation discovery projects, most notably the SNP Consortium, fail to satisfy this requirement because only a small and often unstratified population is screened rendering it impossible to discover the rare variants existing under the multi-equivalent risk model. The multi-equivalent risk model has been systematically ignored in nearly all disease allele discovery studies. Instead, there is an overwhelming preference for the common-disease common-variant hypothesis in the “SNP-o-typing” community because it supports the status quo of low allele frequency resolution genotyping.

It is highly unlikely that the common-disease common-variant hypothesis is the only model describing the association between alleles and disease. Therefore, there is an obvious need for high throughput, post-genomic technologies that resolve both common and rare alleles in a panel of several thousand individuals, a task difficult to perform with current DNA sequencing tools due to time and cost considerations.

One high throughput, post-genomic technology is “MALDI-on-a-chip” mass spectroscopy. The technology uses matrix assisted laser desorption ionization time-of-flight mass spectroscopy (MALDI-TOF MS) to perform point mutation genotyping. The technique not only analyzes the source genomic DNA, it also detects SNPs as the product of allele discrimination reactions. The MALDI procedure calls for the amplification of a piece of the queried genomic DNA that includes the SNP followed by manipulation of the product to reduce mass fragment size during analysis. The advantage of using mass spectroscopy or MS for genotyping studies is that the technique is highly sensitive, yields highly reproducible data, and can reliably distinguish between the most indistinguishable phenotypes such as A/T heterozygotes. MS genotyping represents one of many methods to validate SNIDE using a high throughput genotyping technology. Others include restriction fragment analyses, pyrosequencing, and oligonucleotide array technologies.

The present invention may be used to predict rare and undetected SNPs from sequence context found to cause common diseases. Depending on the genes or the dataset used for determining the gene mutation predictiveness matrix of the present invention, allelic variants account for a substantial proportion of the population risk in a usefully predictive way. In order to use high throughput genotyping for SNP discovery, the present inventors identified the locations of the genome to build a gene mutation predictiveness matrix. One such location for targeting was based on the observation that arginines were frequently involved in disease-causing mutations, particularly when associated with CpG islands. Methylated cytosine (5 mC) spontaneously deaminates to thymine at a high rate. Four of the six possible codons for arginine, CGT, CGC, CGA, and CGG, contain CpGs that may undergo a transition to TpG or CpA (due to a 5 mC to thymine transition on the antisense strand followed by a miscorrection of G to A on the sense strand), which may generate nonsense or missense mutations. To determine if there are other such trends, a systematic study of all disease-causing human mutations was undertaken. One source for mutation data is the Human Genome Mutation Database (HGMD), a non-redundant catalog of 21,541 disease-causing germline human genetic mutations culled from published studies in 1042 genes, 12,858 of which are nonsynonymous point mutations. The HGMD is manually curated and only details mutations that are known to cause a disease. Because only mutations that are known to cause a disease are in the dataset, the aggregate mutation set is biased towards these “phenogenic” mutations that display a clinically realizable phenotype. The large number of different genes analyzed ensures that these biases are not private characteristics of one particular gene but a global property of all loci in the database. In fact, 64% of the loci detailed have 10 or fewer mutations reported. Such characteristics sets the HGMD apart from other variation databases such as dbSNP, HGBASE, and the European Bioinformatics Institute's HUMUT, which often includes many variants whose relationship to disease are unknown. HGBASE is the best annotated of this set but it only describes 3,146 nonsynonymous mutations, the vast majority of which are included in the HGMD. The HGMD was originally established for the study of the mechanisms of human mutation, but has developed into a centralized resource of broad utility to researchers, physicians, and genetic counselors. Therefore, the HGMD is the premier database to study the relationship between mutation type and clinical impact.

Statistical Analysis of Mutation Frequency. A statistical analysis was undertaken of the HGMD data revealing that point mutations share contextual sequence features. The mutations were grouped into classes that are defined by the wild-type and mutant codon pair such “CGA→CAA”. There are a total of 3*3*3*64=576 of these classes possible, of these there are 424 codon mutation classes out of the possible 576. Of those classes that are not seen, 14 are rare and 138 are silent. For each mutation class, a predictive value derived from the HGMD data was defined that encompasses: 1) the likelihood that a given point mutation will occur; and 2) the impact of that mutation. For any given class, this predictiveness value, ζ, is that class's frequency in the HGMD, which may be further weighted by codon usage to correct for the fact that certain classes may appear to be frequent only because the wild type usage is high. These values are then normalized to 100. TABLE 1A lists the twenty classes most and least predictive of disease as determined by ζ. It is not surprising that most of the highly predictive mutation classes in TABLE 1A and 1B occur at CpG dinucleotides that are known to be highly prone to mutation (methylated cytosine spontaneously deaminate to thymine). TABLE 1B is a complete listing of a codon predictiveness matrix according to one embodiment of the present invention.

TABLE 1A
Codon Mutation Classes Exhibit a 2000-fold Range in Predictiveness (ζ)
of Causing Disease

| Twenty Most Predictive Mutation Classes | | | | | Twenty Least Predictive Mutation Classes | | | | |
|---|---|---|---|---|---|---|---|---|---|
| | Wild- | | Wild-type | Mutant | | Wild- | | Wild-type | Mutant |
| | type | Mutant | Amino | Amino | | type | Mutant | Amino | Amino |
| ζ | Codon | Codon | Acid | Acid | ζ | Codon | Codon | Acid | Acid |
| 9.90 | CGA | TGA | Arg | Stop | 0.0052 | ACC | AGC | Thr | Ser |
| 2.51 | CGG | TGG | Arg | Trp | 0.0053 | CTC | ATC | Leu | Ile |
| 2.48 | CGC | TGC | Arg | Cys | 0.0069 | TCT | GCT | Ser | Ala |
| 2.43 | CGT | TGT | Arg | Cys | 0.0085 | CAA | CAT | Gln | His |
| 2.08 | CGT | CAT | Arg | His | 0.0116 | TCC | GCC | Ser | Ala |
| 1.74 | CGA | CAA | Arg | Gln | 0.0116 | TCC | ACC | Ser | Thr |
| 1.73 | ACG | ATG | Thr | Met | 0.0120 | TTT | TTA | Phe | Leu |
| 1.73 | CGG | CAG | Arg | Gln | 0.0124 | AAG | ATG | Lys | Met |
| 1.71 | CGC | CAC | Arg | His | 0.0127 | TAC | TTC | Tyr | Phe |
| 1.66 | CCG | CTG | Pro | Leu | 0.0127 | AAA | AAC | Lys | Asn |
| 1.51 | TGG | TAG | Trp | Stop | 0.0128 | ATT | CTT | Ile | Leu |
| 1.45 | CAG | TAG | Gln | Stop | 0.0136 | GCG | TCG | Ala | Ser |
| 1.36 | TGG | TGA | Trp | Stop | 0.0137 | ACA | TCA | Thr | Ser |
| 1.33 | TCG | TTG | Ser | Leu | 0.0141 | ATA | CTA | Ile | Leu |
| 1.15 | CAA | TAA | Gln | Stop | 0.0145 | GTA | TTA | Val | Leu |
| 1.06 | GGG | AGG | Gly | Arg | 0.0145 | CCG | ACG | Pro | Thr |
| 1.05 | TGT | TAT | Cys | Tyr | 0.0148 | CAG | CTG | Gln | Leu |
| 0.99 | TGT | CGT | Cys | Arg | 0.0148 | TTC | TAC | Phe | Tyr |
| 0.93 | GGA | AGA | Gly | Arg | 0.0156 | ACC | TCC | Thr | Ser |
| 0.89 | GGT | GAT | Gly | Asp | 0.0160 | CTT | ATT | Leu | Ile |

TABLE 1B
Codon Mutation Classes Exhibit a 2000-fold Range in
Predictiveness of Causing Disease*

| | | | | | |
|---|---|---|---|---|---|
| 9.91 | CGA | TGA | 0.37 | TAT | TAG |
| 2.51 | CGG | TGG | 0.37 | ATG | GTG |
| 2.48 | CGC | TGC | 0.36 | CGG | CCG |
| 2.44 | CGT | TGT | 0.36 | GGT | CGT |
| 2.08 | CGT | CAT | 0.36 | CGC | CTC |
| 1.75 | CGA | CAA | 0.35 | AAT | AGT |
| 1.73 | ACG | ATG | 0.34 | CTC | CCC |
| 1.73 | CGG | CAG | 0.34 | TCA | TAA |
| 1.72 | CGC | CAC | 0.33 | CCC | CTC |
| 1.66 | CCG | CTG | 0.33 | TAT | TAA |
| 1.51 | TGG | TAG | 0.32 | GGA | GTA |
| 1.45 | CAG | TAG | 0.31 | TGC | GGC |
| 1.35 | TGG | TGA | 0.31 | CGT | AGT |
| 1.34 | TCG | TTG | 0.31 | CGT | CTT |
| 1.15 | CAA | TAA | 0.31 | CGA | CTA |
| 1.06 | GGG | AGG | 0.30 | CTT | CCT |
| 1.05 | TGT | TAT | 0.30 | TTA | TGA |
| 0.98 | TGT | CGT | 0.29 | GGC | GTC |
| 0.93 | GGA | AGA | 0.29 | TAA | TAT |
| 0.89 | GGT | GAT | 0.29 | TCC | TTC |
| 0.89 | GGT | AGT | 0.29 | CAC | TAC |
| 0.87 | GCG | GTG | 0.29 | GCC | ACC |
| 0.78 | TAT | TGT | 0.28 | TGC | TGG |
| 0.75 | TGC | TAC | 0.28 | GGA | TGA |
| 0.66 | TGC | CGC | 0.28 | GAA | AAA |
| 0.63 | GGG | GAG | 0.27 | GCG | GAG |
| 0.63 | TAC | TAA | 0.27 | TTA | TAA |
| 0.63 | TCA | TGA | 0.27 | TGT | TGA |
| 0.63 | TGG | CGG | 0.27 | TGG | TGT |
| 0.60 | GGA | GAA | 0.26 | GCT | ACT |
| 0.60 | GGC | AGC | 0.25 | TTA | TCA |
| 0.60 | GGT | GTT | 0.25 | CTC | TTC |
| 0.59 | GGC | GAC | 0.25 | ATA | ACA |
| 0.59 | TCG | TAG | 0.25 | TGA | AGA |
| 0.55 | TGC | TGA | 0.25 | TGA | TGT |
| 0.53 | CAT | CGT | 0.25 | GAT | GGT |
| 0.53 | TAC | TAG | 0.25 | TCG | CCG |
| 0.51 | CGT | CCT | 0.24 | GCG | ACG |
| 0.48 | CTG | CCG | 0.24 | TCC | CCC |
| 0.47 | ATT | ACT | 0.24 | TGG | TGC |
| 0.46 | GGT | TGT | 0.24 | GCA | ACA |
| 0.46 | CTA | CCA | 0.24 | TAC | CAC |
| 0.46 | GAG | AAG | 0.24 | CCT | TCT |
| 0.45 | GTG | ATG | 0.23 | TGC | TCC |
| 0.45 | GAA | TAA | 0.23 | TTG | TCG |
| 0.44 | CGA | GGA | 0.23 | TGT | TCT |
| 0.41 | TAC | TGC | 0.23 | CAT | TAT |
| 0.40 | ATG | ACG | 0.22 | TCT | CCT |
| 0.40 | CGC | CCC | 0.22 | TGG | GGG |
| 0.39 | CGA | CCA | 0.22 | CCC | TCC |
| 0.38 | GAC | AAC | 0.22 | CCG | CGG |
| 0.38 | GAG | TAG | 0.22 | GGG | CGG |
| 0.37 | TGC | TTC | 0.21 | CCA | CTA |
| 0.21 | GAC | GGC | 0.15 | TAA | GAA |
| 0.21 | CAC | CGC | 0.14 | CTT | CGT |
| 0.21 | GCC | GTC | 0.14 | GAC | CAC |
| 0.21 | CGG | GGG | 0.14 | AGA | TGA |
| 0.21 | ACG | AGG | 0.14 | AAG | GAG |
| 0.21 | TTT | TCT | 0.14 | ATA | AAA |
| 0.21 | ACC | ATC | 0.14 | CTG | CGG |
| 0.21 | TGT | TTT | 0.14 | TGG | AGG |
| 0.21 | GTT | TTT | 0.14 | GAT | GTT |
| 0.21 | ATG | ATA | 0.14 | GTT | ATT |
| 0.20 | TAG | CAG | 0.14 | TAC | GAC |
| 0.20 | CGT | GGT | 0.14 | GAC | GTC |
| 0.20 | ATG | AGG | 0.14 | TCA | TTA |
| 0.20 | AGT | AAT | 0.14 | AGG | GGG |
| 0.19 | GTC | ATC | 0.13 | TAT | CAT |
| 0.19 | CGG | CTG | 0.13 | TAT | GAT |
| 0.19 | GCT | GTT | 0.13 | TTC | CTC |
| 0.19 | CCT | CTT | 0.13 | GAC | TAC |
| 0.19 | GCT | CCT | 0.13 | GCT | GAT |
| 0.19 | GTC | TTC | 0.13 | CGC | GGC |
| 0.19 | GAT | AAT | 0.13 | AAT | GAT |
| 0.19 | TGT | GGT | 0.13 | GTA | ATA |
| 0.18 | GCC | GAC | 0.13 | CAC | CAG |
| 0.18 | GGC | CGC | 0.13 | ACG | AAG |
| 0.18 | TGG | TCG | 0.13 | ATG | AAG |
| 0.18 | AGA | GGA | 0.13 | CCC | CGC |
| 0.18 | AAC | AAG | 0.13 | AGG | AAG |
| 0.18 | ACA | ATA | 0.12 | TCT | TTT |
| 0.17 | TGT | TGG | 0.12 | GGA | GCA |
| 0.17 | GTC | GAC | 0.12 | GTT | GAT |
| 0.17 | AAC | AGC | 0.12 | AGC | AAC |
| 0.17 | TTC | TCC | 0.12 | CTT | TTT |
| 0.17 | ATC | ACC | 0.12 | AAA | GAA |
| 0.17 | TGA | TCA | 0.12 | CAT | CCT |
| 0.17 | TGA | TGG | 0.12 | CAT | CTT |
| 0.17 | TGA | GGA | 0.12 | CAT | CAG |
| 0.17 | CGC | AGC | 0.12 | GTA | GGA |
| 0.17 | GGC | TGC | 0.12 | GCA | GAA |
| 0.17 | GCA | GTA | 0.12 | TGC | AGC |
| 0.17 | ATC | AAC | 0.12 | GTG | GCG |
| 0.17 | ACT | ATT | 0.11 | ATA | ATG |
| 0.16 | AAC | AAA | 0.11 | GAA | GGA |
| 0.16 | GGG | TGG | 0.11 | GGG | GTG |
| 0.16 | CTC | CGC | 0.11 | CAC | CCC |
| 0.16 | TCG | TGG | 0.11 | GAT | CAT |
| 0.16 | TTG | TAG | 0.11 | GAT | TAT |
| 0.15 | AAA | TAA | 0.11 | AAG | TAG |
| 0.15 | TTT | TGT | 0.11 | TAC | AAC |
| 0.15 | CAG | CGG | 0.11 | TTG | TTC |
| 0.15 | TCA | CCA | 0.11 | AGA | AAA |
| 0.15 | TAA | CAA | 0.10 | GGA | CGA |
| 0.15 | GTA | GCA | 0.10 | ACC | CCC |
| 0.15 | TAA | AAA | 0.10 | GCC | CCC |
| 0.10 | GGT | GCT | 0.08 | GTG | TTG |
| 0.10 | ACT | CCT | 0.08 | CCT | CAT |
| 0.10 | TGT | AGT | 0.08 | CCT | ACT |
| 0.10 | GTT | GCT | 0.08 | AGT | AGA |
| 0.10 | ATC | GTC | 0.08 | ACA | AAA |
| 0.10 | CCG | TCG | 0.08 | ACA | AGA |
| 0.10 | CCG | CAG | 0.07 | GTT | CTT |
| 0.10 | AGC | AGA | 0.07 | TTG | TTT |
| 0.10 | CCT | CGT | 0.07 | TTG | TGG |
| 0.10 | ATA | AGA | 0.07 | CTA | GTA |
| 0.10 | ATA | GTA | 0.07 | ACC | GCC |
| 0.10 | GTG | GAG | 0.07 | GTA | GAA |
| 0.10 | CAG | CCG | 0.07 | CCT | GCT |
| 0.10 | ATC | ATG | 0.07 | ATT | TTT |
| 0.10 | ATC | TTC | 0.07 | ATT | AGT |
| 0.10 | TCT | TGT | 0.07 | GTG | CTG |
| 0.10 | AAC | GAC | 0.07 | TTC | TGC |
| 0.10 | GCA | CCA | 0.07 | CAC | CAA |
| 0.10 | TAC | TCC | 0.07 | CAT | CAA |
| 0.10 | GCG | CCG | 0.07 | ATG | ATT |
| 0.09 | AGC | AGG | 0.07 | AAC | ATC |
| 0.09 | TTC | GTC | 0.07 | AAC | ACC |
| 0.09 | AGT | GGT | 0.07 | GTT | GGT |
| 0.09 | AGT | AGG | 0.07 | ATC | AGC |
| 0.09 | AGT | ATT | 0.06 | TCC | TAC |
| 0.09 | CCA | CAA | 0.06 | ATG | TTG |
| 0.09 | TAT | TCT | 0.06 | AGG | AGT |
| 0.09 | CCC | CAC | 0.06 | AGG | AGC |
| 0.09 | TTT | CTT | 0.06 | AGC | GGC |
| 0.09 | AGA | ACA | 0.06 | AGA | AGT |
| 0.09 | GGC | GCC | 0.06 | GTC | CTC |
| 0.09 | ACC | AAC | 0.06 | CAC | GAC |
| 0.09 | CAT | GAT | 0.06 | CTG | CAG |
| 0.09 | GTG | GGG | 0.06 | CCA | ACA |
| 0.09 | ACT | GCT | 0.06 | GAG | GAC |
| 0.09 | TCC | TGC | 0.06 | CAA | AAA |
| 0.09 | AAC | TAC | 0.06 | AAT | CAT |
| 0.09 | CCA | TCA | 0.06 | AGT | CGT |
| 0.08 | TGA | TTA | 0.06 | TAT | AAT |
| 0.08 | TGA | CGA | 0.06 | CTC | CAC |
| 0.08 | TGA | TGC | 0.06 | CTC | GTC |
| 0.08 | ATT | GTT | 0.06 | GAG | CAG |
| 0.08 | GTC | GGC | 0.06 | ATT | AAT |
| 0.08 | ACA | GCA | 0.06 | ATT | ATG |
| 0.08 | AGG | ACG | 0.06 | CAG | GAG |
| 0.08 | CCC | ACC | 0.06 | CAG | CAC |
| 0.08 | ACG | GCG | 0.06 | AAG | AAC |
| 0.08 | ACG | CCG | 0.06 | GTC | GCC |
| 0.08 | GGG | GCG | 0.06 | CAC | AAC |
| 0.08 | TTC | TTA | 0.05 | AGG | TGG |
| 0.08 | TTC | TTG | 0.05 | GAC | GAG |
| 0.08 | GAG | GGG | 0.05 | TTT | GTT |
| 0.08 | TGG | TTG | 0.05 | TTT | ATT |
| 0.05 | CTG | GTG | 0.03 | GTA | CTA |
| 0.05 | AGA | AGC | 0.03 | TTA | ATA |
| 0.05 | AAT | AAG | 0.03 | TTA | TTC |
| 0.05 | AAG | AGG | 0.03 | ATA | TTA |
| 0.05 | AGC | CGC | 0.03 | TTA | TTT |
| 0.05 | AGC | ATC | 0.03 | CTG | ATG |
| 0.05 | CAA | CGA | 0.03 | GAA | GCA |
| 0.05 | AAA | AGA | 0.03 | AAG | CAG |
| 0.05 | GCC | TCC | 0.03 | GCT | TCT |
| 0.05 | TTG | GTG | 0.03 | GCT | GGT |
| 0.05 | CTT | CAT | 0.03 | GAT | GAG |
| 0.05 | ACT | AGT | 0.03 | GAT | GCT |
| 0.05 | AAT | AAA | 0.03 | GCA | GGA |
| 0.05 | GAC | GAA | 0.03 | CAA | CAC |
| 0.05 | AAG | AAT | 0.03 | AAA | ACA |
| 0.05 | GAA | CAA | 0.03 | AGT | TGT |
| 0.05 | AGG | ATG | 0.03 | AAA | CAA |
| 0.04 | AGA | ATA | 0.02 | GAA | GAC |
| 0.04 | CTA | CGA | 0.02 | TTT | TAT |
| 0.04 | CCA | CGA | 0.02 | ACT | AAT |
| 0.04 | TTT | TTG | 0.02 | ACT | TCT |
| 0.04 | AAT | ATT | 0.02 | AAT | TAT |
| 0.04 | ATG | ATC | 0.02 | TCG | GCG |
| 0.04 | AAC | CAC | 0.02 | TCG | ACG |
| 0.04 | GCG | GGG | 0.02 | AAG | ACG |
| 0.04 | CCC | GCC | 0.02 | AGC | TGC |
| 0.04 | CTT | GTT | 0.02 | GAA | GAT |
| 0.04 | GCC | GGC | 0.02 | TCT | ACT |
| 0.04 | GCA | TCA | 0.02 | TCA | ACA |
| 0.04 | GAA | GTA | 0.02 | CAA | CTA |
| 0.04 | CAG | AAG | 0.02 | AGT | ACT |
| 0.04 | AGC | ACC | 0.02 | AAA | AAT |
| 0.04 | CCA | GCA | 0.02 | TTG | ATG |
| 0.04 | ATG | CTG | 0.02 | CTT | ATT |
| 0.03 | TCT | TAT | 0.02 | ACC | TCC |
| 0.03 | TTC | ATC | 0.01 | CAG | CTG |
| 0.03 | CAC | CTC | 0.01 | TTC | TAC |
| 0.03 | CAA | CCA | 0.01 | CCG | ACG |
| 0.03 | CAA | GAA | 0.01 | GTA | TTA |
| 0.03 | ACA | CCA | 0.01 | ATA | CTA |
| 0.03 | TAT | TTT | 0.01 | ACA | TCA |
| 0.03 | GAG | GAT | 0.01 | GCG | TCG |
| 0.03 | CAG | CAT | 0.01 | ATT | CTT |
| 0.03 | ATC | CTC | 0.01 | AAA | AAC |
| 0.03 | GAT | GAA | 0.01 | TAC | TTC |
| 0.03 | GAC | GCC | 0.01 | AAG | ATG |
| 0.03 | GAG | GTG | 0.01 | TTT | TTA |
| 0.03 | GAG | GCG | 0.01 | TCC | GCC |
| 0.03 | AAT | ACT | 0.01 | TCC | ACC |
| 0.03 | AAA | ATA | 0.01 | CAA | CAT |
| 0.03 | CTA | CAA | 0.01 | TCT | GCT |
| 0.03 | CAT | AAT | 0.01 | CTC | ATC |
| 0.03 | CCG | GCG | 0.01 | ACC | AGC |

*Predictiveness, wild-type codon, mutanta codon shown

Neighboring Nucleotide Effects on Mutation. Although interesting, the data in TABLE 1A provides a first-order analysis. It does not take into account important neighboring nucleotide effects that impact the likelihood of mutation. For example, the mutability of a codon such as GGG would be heavily influenced by a 5′ C which, if methylated, can deaminate to thymine on the antisense strand causing a miscorrection of the G in the first position of the codon to A. A study of GGG to AGG mutations (G→R) shows that a disproportionate fraction of these, the codon preceding the GGG ended with a C. Generally, sequence farther than one base 5′ or 3′ from the mutating base has little effect on the likelihood of mutation. To complete the statistical analysis of mutation data, it is desirable to subdivide these codon mutation classes further by the 5′ and 3′ flanking nucleotide. For classes where the mutation occurs at the second position of a codon, this information is already implicit in the codon identity; however, for mutations in the first and third positions the classes may be subdivided. The HGMD supplies such information. SNIDE may predict pSNPs using either mode (flanking information included or excluded) depending on the application.

One problem with this method is that going from 4³ to 4⁵ “super codons” dilutes the data considerably. To overcome the dilution, only codon mutation classes deemed to have sufficient sample size were subdivided by flanking nucleotide. This sampling affected 21 mutation classes. For example, CGT→TGT mutations cumulatively have a frequency of 2.48%, but when subdivided by flanking nucleotide the frequencies (weighted by usage) are 0.91% for cCGT→TGT, 0.68% for gCGT→TGT, 0.48% for tCGT→TGT, and 0.34% for aCGT→TGT. When weighting this subset of mutation classes by usage, it is no longer appropriate to apply the usage of each codon. Instead, usage of each nXXX or XXXn “super codon” class was directly calculated from, e.g., the UniGene build of human cDNA clusters. Each UniGene cluster contains sequences that represent a unique gene, and the longest sequence from each cluster was chosen for usage calculation. The addition of neighboring nucleotide effects into the mutation statistical analysis increased the total number of mutation classes to 496.

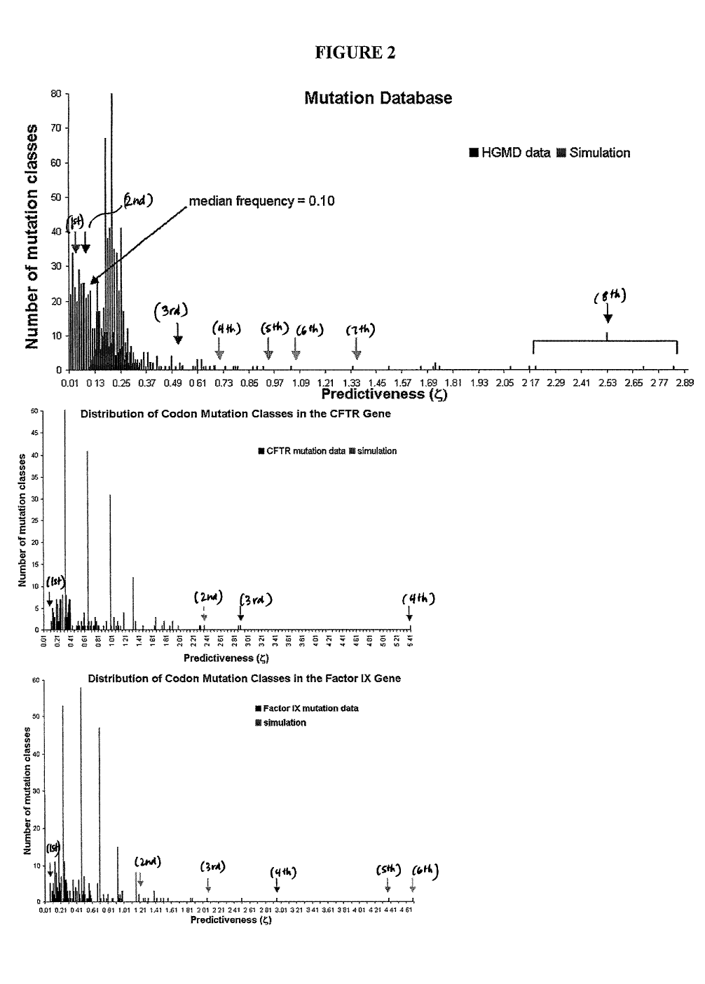

Features of Human Gene Mutation. Disease causing mutation is highly non-random. It was found that the magnitude of difference between predictiveness, ζ, of each mutation class as shown in TABLE 1 and the known mutation sites were different. FIG. 2A-C depicts the distribution of mutations per the 496 mutation classes compared to what would be expected at random, that is, if all mutation classes were equally likely to cause disease. FIG. 2A shows that the mutation data in no way approximates the expected multinomial distribution and clearly demonstrates that there is a considerable set of outliers up to 27 times greater than the median value suggesting that certain mutation classes cause disease much more often than others (for arrows, from left to right: 1^{st}, GAG→GCT; 2^{nd}, GTG→GAG; 3^{rd}, CAA→TAA; 4^{th} GGA→AGA; 5^{th}, TGT→CGT; 6^{th}, TGT→TAT; ₇th, TGG→TAG; 8^{th}, CGA→TGA). In fact, CGA→TGA transitions alone account for 4.76% of all disease-causing alleles in the database and are cumulatively nearly 2000-fold more predictive than the least frequent transition, ACC to AGC (Thr→Ser). There is also as set of mutation classes that are less likely than random to cause disease. These are highly conservative substitutions, as shown in TABLE 1, where four Ile⇄Leu classes are in this set. These distribution characteristics are not dominated by the effects of a few genes because the distributions of smaller sets of randomly picked genes from the HGMD are similar.

FIG. 2 shows the distribution of nonsynonymous codon mutation classes in: (2A) the whole HGMD; (2B) CFTR gene; and (2C) Factor IX gene. The predictiveness of each codon mutation class was calculated as (# of mutations in class)/(total # of mutations in HGMD)/(wild-type codon usage) and normalized to 100. The simulations approximate the distribution if all mutation classes were equally likely to occur in the HGMD, Factor IX gene, or the CFTR gene, which creates multinomial distributions, an extension of the binomial distribution to the case where an attribute has more than two possibilities. FIG. 2A shows that the HGMD (12,858 mutations) can be categorized into 496 codon mutation classes, 84 of which include flanking nucleotide information and are calculated as described herein below. The simulation (1^{st} arrow) was performed as rolling a 496-sided die 12,858 times. Frequencies in the simulation were calculated as (# of times each side of die was found)/(total number of rolls) and normalized to 100. FIG. 2B shows that the CFTR gene (303 mutations) can be categorized into 173 codon mutation classes (for arrows, from left to right: 1^{st}, GTG→GAG; 2^{nd}, TGG→TAG; 3^{rd}, CAA→TAA; 4^{th}, CGA→TGA). The simulation is akin to rolling a 173-sided die 303 times. FIG. 2C shows that mutation frequency for the Factor IX gene (436 mutations) can be categorized into 214 codon mutation classes (for arrows, from left to right: 1^{st}, GAG→GCT; 2^{nd}, TGG→TAG; 3^{rd}, GGA→AGA; 4^{th}, CGA→TGA; 5^{th}, TGT→CGT; 6^{th}, TGT→TAT). The simulation (1^{st} arrow) is akin to rolling a 214-sided die 436 times. The presence of far outliers is the most striking part of all three distributions. Both the CFTR and Factor IX data show extreme, very predictive outliers that mirror the cumulative HGMD distribution. There is also a set of outliers less likely than random to cause disease, as shown by the leftmost arrows in FIGS. 2A-2C: GTG→GAG and GAG→GCT. As the CFTR and F9 examples show, even individual genes approximate the mutational properties of the global mutation class distribution.

Although FIG. 2A describes the global mutation properties of a large set of genes, the hallmarks of the HGMD distribution can still be seen in single gene cases, such as for CFTR and Factor IX (FIGS. 2B-C). For these two genes, the distribution again does not approximate what would be expected at random. The most important feature of all three graphs is the set of outlier mutation classes in the far right portion of the graph. The identity of the outliers is well conserved in each of the graphs, which shows that the most causative mutation classes in a global-sense are identical to the most causative mutation classes on a single gene level. The same may be said of the converse, that the least predictive mutations in the single gene distributions double as the least predictive mutation classes in the entire body of disease causing mutation.

Development of SNIDE, A Method and System for Single Nucleotide Variation IDEntification. The present inventors recognized that data in FIGS. 2A-C indicated that certain codons are especially mutagenic and causative and therefore represent the best targets to query when looking for gene mutations associated with any disease. FIGS. 2A-2C indicated that predictions of phenogenic variation in a gene were possible. Next, the inventors determined the level of accuracy of those predictions. The predictive nature of all disease causing mutation data has been incorporated into the computational method and system SNIDE (Single Nucleotide variation IDEntification), which predicts variants using the following steps: (1) input of each codon in a queried DNA sequence; (2) determination of each possible nonsynonymous mutation; (3) assignment of predictiveness to that mutation based on the identity of the wild-type and resultant codon; and (4) ranking of all predictiveness values to highlight the most probable mutations in the gene. All input sequences may be filtered for low complexity regions because such regions are expected to be highly variable and prone to many contraction and expansion polymorphisms with modest or negligible effects on health.

The predictiveness values are the predictiveness of the mutation class caused by the codon mutation, such as those seen in TABLE 1. For example, a CGA (Arg) codon in a queried sequence could point mutate to TGA, AGA, GGA, CTA, CAA, CCA, CGT, CGC, or CGG. Five of these point mutations are silent, but the rest can be assigned a predictive value based on the ζ-value in the distribution (FIG. 2A). The SNIDE method may also accept a user-defined threshold that describes how much of the right tail of the distribution in FIG. 2A should be used as predictive information. For example, to only scan a DNA sequence for predictions corresponding to the fifty farthest outliers in FIG. 2A, the user would enter a value of 50/496=10% (only consider the top 10% most predictive mutation classes). A threshold of 100% would cause all possible nonsynonymous predictions to be made.

SNIDE is also useful for predicting point mutations in a wild-type sequence that will cause a phenotypic mutation based on a mutant gene dataset, e.g., the HGMD data. SNIDE predicts point mutation sites for directed high-throughput genotyping that, at a rate superior to random, will be associated with disease due to a predictable mutation. No technology, other than SNIDE, allows the user to genotype a large sample size for novel and or suspected SNPs, in particular for those cases where the members of the samples are not aware of a SNP phenotype.

Thus far, SNIDE has predicted causative variation (pSNPs), but the statistical methods used to generate the predictive matrix can be mirrored to predict pharmacologic irrelevant neutral variation, herein known as nSNPs.

The procedural difference in composing an nSNP matrix lies in the choice of mutation database for matrix training. To discover pSNPs, HGMD was used for training and a neutral variation source, e.g., NCBIs dbSNP, was used for discovery. dbSNP is generated from primarily low-pass sequencing studies in a small number of healthy yet ethnically dissimilar individuals while the HGMD is a carefully curated depository of mutations gleaned from peer-reviewed journals that are deemed to possess significant evidence of phenotype causation. If, in fact, the profile of pSNPs is separate from the profile of mutations that are not causative (nSNPs), a comparison of dbSNP and the HGMD should yield significant differences. Obviously, the HGMD will not include synonymous mutations. Because the SNIDE matrix merely ranks all possible codon to codon mutation classes (e.g., CGA→TGA) by their likelihood of existing somewhere in the population, a comparison of the ranks of each codon mutation class between the nSNP and pSNP matrixes will detail the differences between neutral variation and deleterious mutation. This is confirmed because the HGMD matrix has nonsense and chemically nonconservative mutation classes at the top of the list while the dbSNP matrix ranks synonymous and conservative amino acid replacements higher than chemically nonconservative mutation classes. It is, therefore, often important to run both pSNP and neutral variation discovery scripts on each gene to be examined for mutations. The reason for running both scripts is twofold: (i) the underlying statistical method of SNIDE can be validated by use of the dbSNP matrix and although it will not find a preponderance of pSNPs, the identical statistical method will have been used to discover the neutral variants that occur at an elevated frequency; and (ii) an nSNP predictive method allows for an estimation of how many neutral variants may be found in a high throughput genotyping study and may aid in the technical aspects of the experiment, such as in primer design.

The novel component of SNIDE does not depend on the database used; rather, it hinges on the statistical methodology employed. SNIDE, as a method, represents the ability to create a matrix of mutation classes ranked by predictiveness dependent on the global properties of any mutation database. As a result, predictive metrics using the statistical analysis of the present invention may be used on all current and future mutation databases.

Evaluation of the SNIDE Point Mutation Prediction Method, System and Algorithm. If the SNIDE prediction method is valid, then predictions from SNIDE analysis will match the pSNPs of well-characterized genes for which there are known, causative variants. The accuracy rate may be estimated (fraction of predicted alleles that are already known to cause disease) and the completeness rate determined (fraction of total known alleles that have been predicted for a gene). A definition for predictiveness threshold is meaningful, and bears an inverse relationship with the accuracy rate. Predictiveness determinations suffer from the fact that not all of the alleles that cause disease in man are known; therefore, accuracy rates will be generally a lower estimate.

SNIDE analysis was performed against the coding DNA of eight human genes (p53, CFTR, hemoglobin-β, connexin 32, von hippel-lindau disease tumor suppressor protein, omithine transcarbamylase, phenylalanine hydroxylase, and Factor IX), having 230, 314, 235, 145, 152, 127, 262, and 436 known phenotype-causing mutations, respectively, according to the HGMD and SWISS-PROT database.

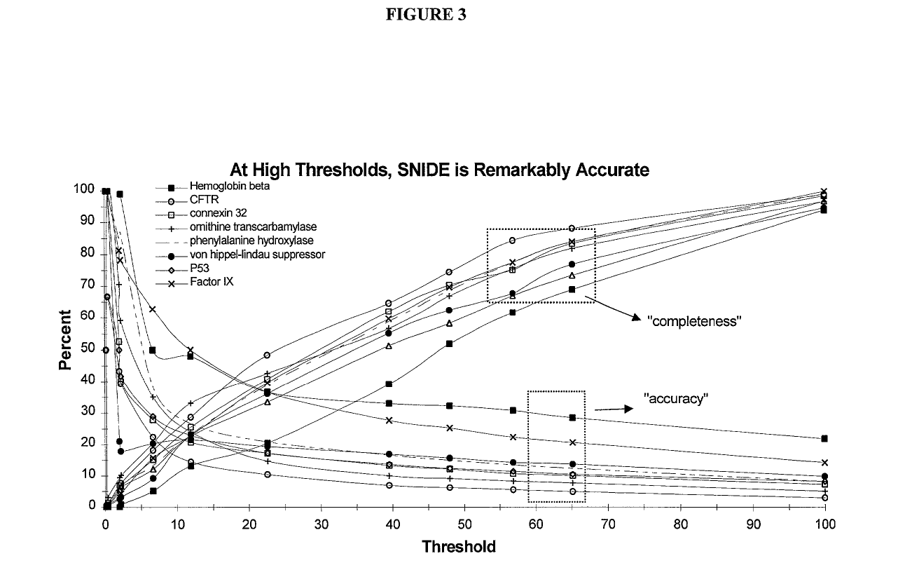

FIG. 3 is a graph that demonstrates the computational validation of SNIDE point mutation predictions. As a function of threshold, FIG. 3 shows the completeness and accuracy of predictions based on the spectrum of known mutations for these genes. Nonsynonymous phenotype-inducing point mutation data for eight well-studied disease-causing genes was collated from the HGMD and SWISS-PROT database. For each gene, a pair of curves (demarcated by dashed boxes) were generated with data points at each possible user-defined threshold (lower number is more selective). The “accuracy” set refers to the percentage of predictions for which causative alleles are known and the “completeness” curve set refers to the percentage of all known causative alleles found by SNIDE. The accuracy rates at the 5% threshold and known number of causative mutations per amino acid (or AA) are: hemoglobin β, 58.3%, 1.6 mutations/AA; Factor IX, 69.57%, 0.9 mutations/AA; von hippel lindau suppressor, 18.0%, 0.7 mutations/AA; phenylalanine hydroxylase, 48.27%, 0.6 mutations/AA; P53, 35.1%, 0.6 mutations/AA; connexin 32, 32.8%, 0.5 mutations/AA; ornithine transcarbamylase, 42.0 %, 0.4 mutations/AA; CFTR, 28.8%, 0.2 mutations/AA. In general, the accuracy of SNIDE is roughly proportional to the number of known, causative mutations per amino acid in the queried gene.

FIG. 3 also demonstrates that at a threshold of 5% (a lower number is more selective), where point mutations predictions were made using only the 25 most predictive mutation classes, the eight genes have accuracy and completeness values ranging from 18.0%/5.92% (von hippel lindau suppressor) to 69.6%/11.5% (Factor IX). Gene to gene differences in accuracy are largely reflected in how thoroughly a gene has been studied, that is, the more people genotyped, the more alleles found. If point mutation predictions were made at random, the accuracy rate for Factor IX would be (436 known mutations)/(461*3 nucleotides in the gene)*(¼ chance of picking the correct mutation)=7.9%, an 8.8-fold worse accuracy statistic. At the 5% threshold, SNIDE performs anywhere from 3.02 (von hippel-lindau suppressor) to 16.94 (CFTR)-fold better than the “at random” prediction method, the average being 9.14. More stringent thresholds are even more impressive. For example, running the eight gene set through a SNIDE analysis at a threshold of 1% (predicting only CGA to TGA mutations) shows that cumulatively 24 out of 27 (88.9%) predictions are already known to cause disease. This is a 19.7-fold improvement over making predictions at random. Furthermore, the remaining three mutations may exist as a causative allele for some disease, but simply have not yet been discovered, or may even be lethal.

The SNIDE algorithm may not necessarily predict all the possible mutations, but rather, likely mutations, e.g., CGA to TGA transitions. In combination with new genotyping techniques the SNIDE predictive algorithm permits speedy discovery of rare, highly causative alleles known to exist under the multi-equivalent risk model. In conjunction with high throughput genotyping, SNIDE analysis may generate results from a large number of genetic tests. For example, women of any age with a familial history of breast cancer may have a blood test done that will screen for approximately 100 causative SNPs in BRCA1, breast cancer 1 gene. This collection of SNPs represents years of research, and the mutation screening test gives individuals invaluable knowledge of their genetic predisposition to disease at any age so that preventative steps may be taken. SNIDE analysis may also aid geneticists in creating these mutation screens more quickly so that one's risk of a variety of diseases may be better understood.

As a point mutation prediction tool, SNIDE can identify likely disease-causing mutations. A codon mutation predictiveness matrix that correlates a predictiveness value: ζ-values for each codon mutation class was developed was designed for gene mining, e.g., the HGMD database, the dbSNP database, disease databases or other human and non-human databases. The results for the predictiveness value are like those in TABLE 1. The SNIDE package may be an assembly of, e.g., three PERL scripts connected by UNIX c-shell (csh) that performs one or more of the following tasks: (1) parsing of either user-supplied genbank or fasta input files delineating the coding DNA to be analyzed; (2) calculation of expected point mutation probabilities according to a user-defined threshold (default=top 5% of all codon mutation classes); and (3) ranking of point mutation predictions by ζ-value and generation of a tab-delimited file suitable for standard spreadsheet applications such as Excel.

The SNIDE algorithm may be tested by making point mutation predictions in a set of genes thought to be associated with a complex disorder that also has a significant patient population and from which a large number of causative mutations have already been identified, such as cardiac disease. The predictions are further tested using high throughput technology such as MS. For example, heart disease patient DNA samples obtained from clinical study may be used, especially because, with a large enough sample group, factors such as genetic diversity, heritability and the number of genes involved can be overcome. For cardiac disease, in which disease definition and actual diagnosis may vary, using a population stratified by many different phenotypes of heart disease may be used. Part of SNIDE's utility is the ability to predict numerous, rare, causative variants that would be missed by mere empirical or “low-pass” mutation discovery methods.

Using cardiac disease as an example, pSNP SNIDE may be run on cardiac candidate genes to get a lower-bound estimate of SNIDE's predictive power relative to random. Given that the “total number of known mutations” statistic for each of these genes is known, an improved method to estimate pSNP SNIDE's predictive power over random was developed. The product of the percent accurate and percent complete statistics were used to creates a new value that describes each method's ability to: a) predict accurately; and b) find all known causative mutations. Initially, this inquiry may seem redundant, but it is possible (and sometimes the case) that the random method has better completeness statistics than SNIDE simply because it makes a larger number of predictions. For example, if a mutation prediction was made at every DNA base in a gene, a completeness rate of 100% would be expected but the accuracy would be quite poor.

TABLE 2 gives the results for four genes examined in this way. Mock genotyping data was constructed by generating both SNIDE and random predictions at a threshold of 5%. The statistics for “at random” predictions were generated for ten trials from a randomized SNIDE matrix and averaged. Any predicted polymorphic position (either using SNIDE or random predictions) that is known to be cardiac-disease associated will be scored as correct. The accuracy rates were surprising given that these genes have not been sequenced in large populations, which is the case for hemoglobin, Factor IX, and CFTR. Most striking is the “ratio” column (ratio of SNIDE % complete* % accurate statistic to “at random” % complete * % accurate statistic) that shows that SNIDE predicts mutations on average 21-fold better than random for these four genes.

Minimally, the variants in TABLE 2 would be found in a cardiac disease-associated study as long as the population was properly stratified. Given that the nature of the SNP-hunting community's low-pass genotyping efforts up-weight the probability of only finding common SNPs, however, it was expected that many new, less common alleles for these genes in the high-pass study would be found, and thus, the ultimate accuracy rate should be considerably greater.

TABLE 2
SNIDE Predicts Mutations Considerably Better Than Random in
Four Cardiac Genes

| | Total | SNIDE predicitions | | | | | | | |
|---|---|---|---|---|---|---|---|---|---|
| Gene | alleles | % accurate | | | | Random predictions | | | |
| Symbol | known | (number/total) | | % complete | Product | % accurate | % complete | Product | Ratio |
| MYH7 | 28 | 3.1 | (7/228) | 60.7 | 188.2 | 0.48 | 17.7 | 8.5 | 22.1 |
| TNNT2 | 9 | 12.5 | (5/40) | 55.6 | 695.0 | 0.76 | 34.6 | 26.3 | 26.4 |
| SCN5A | 8 | 1.6 | (5/308) | 62.5 | 100.0 | 0.15 | 27.3 | 4.1 | 24.3 |
| KCNQ1 | 44 | 12.3 | (17/138) | 38.6 | 474.9 | 2.16 | 16.9 | 36.5 | 13.1 |

MYH7 = myosin heavy polypeptide 7, cardiac muscle beta;
TNNT2 = cardiac troponin T2;
SGN5A = sodium channel voltage gated protein type V, alpha polypeptide;
KCNQ1 = potassium voltage gated channel KQT-like subfamily member 1.
% complete refers to the percentage of known disease alleles predicted.
“Product” is the product of % complete and % accurate statistics.
Ratio refers to (% complete × % accurate of SNIDE)/(% complete × % accurate of randomly choosing sites of causative variation) and is a measure of how much fold better SNIDE predicts than random.

Validation of the SNIDE algorithm by DNA sequencing is important. A population consisting of 132 patients with dilated cardiomyopathy and 60 cancer cell lines was acquired. Candidate genes for dilated cardiomyopathy were collated through a extensive literature review, as shown in TABLE 3.

TABLE 3
Candidate Dilated Cardiomyopathy Genes

| | Gene | NCBI accession | Region | |
|---|---|---|---|---|
| Gene | name | number | amplified | Literature data |
| bradykinin b2 | BDKRB2 | S45489 | 272-871 | Dilation of left ventricle in |
| receptor | | | | −/− knockout mice[1] |
| endothelin-A | EDNRA | D11145 | 79-496 | Ralph Shohet |
| receptor | | | | |
| beta | ADRB1 | AL355543 | 36118- | Subpopulation of idiopathic |
| adrenergic | | | 36750 | DCM patients demonstrate |
| receptor 1 | | | | auto-antibodies against the |
| | | | | protein product[2] |
| beta | ADRB2 | Y00106 | 1287- | G-protein coupled receptor |
| adrenergic | | | 1868 | that may be involved in a |
| receptor 1 | | | | signaling pathway with |
| | | | | CREB.[3] |
| CREB1 | CREB1 | 10716632 | 133373- | Transgenic mice expressing |
| | | | 133787 | CREB under the control of |
| | | | | a cardiac myocyte-specific |
| | | | | alpha myosin heavy chain |
| | | | | promoter developed |
| | | | | DCM.[3] |
| MCIP | MCIP | 7768679 | 109773- | Expression of MCIP is |
| | | | 110435 | regulated by calcineurin |
| | | | | which modulates gene |
| | | | | expression in cardiac |
| | | | | muscle[4] |

[1]Emanueli et. al. Dilated and Failing Cardiomyopahty in Bradykinin B2 Receptor Knockout Mice. Circulation. 1999;100:2359-2365;
[2]Magnusson et. al. Mapping of a functional autoimmune epitope on the beta 1-adrenergic receptor in patients with idiopathic dilated cardiomyopathy. J. Clin. Invest. 1990;86: 1658-63.
[3]Fentzke et. al. Evaluation of ventricular and arterial hemodynamics in anesthetized closed-chest mice. J Am Soc Echocardiogr. 1997;10(9):915-25.
[4]Yang et. al. Independent signals control expression of the calcineurin inhibitory proteins MCIP1 and MCIP2 in striated muscles. Circ Res. 2000;87:E61-8.

Each gene was run through SNIDE using the HGMD (causative mutation) matrix to select the most pSNP-heavy 500-600 bp coding DNA region for dye-terminator sequencing using the Beckman CEQ-2000XL. TABLE 4 shows the current sequencing status, and TABLES 5, 6 and 7 detail some of the mutations that have been discovered.

TABLE 4
Data Analysis Status of DCM Association Study

| | | | | | Reverse | Final |
|---|---|---|---|---|---|---|
| Gene | PCR | Sequencing | Sequencing | Sequences | reads | mutation list |
| name | optimized? | Optimized? | completed? | analyzed? | completed | constructed |
| BDKRB2 | Yes | Yes | Yes | Yes | Yes | Yes |
| EDNRA | Yes | Yes | Yes | Yes | Yes | Yes |
| ADRB1 | Yes | Yes | Yes | Yes | Yes | No |
| ADRB2 | Yes | Yes | Yes | Yes | No | No |
| CREB1 | Yes | Yes | Yes | Yes | No | No |
| MCIP | Yes | Yes | Yes | Yes | No | No |

TABLE 5
SNPs Discovered in BDKRB2

| | | | | | | | Codon |
|---|---|---|---|---|---|---|---|
| Codon | Amino | Genotype | | | | | mutation |
| mutation | acid | (number | | | SNIDE | Matrix | class rank |
| class | change | individuals) | Position* | Novel? | prediction? | type | in matrix |
| ACG→ACA | Thr→Thr | G/G(159) | 792 | No | Yes | dbSNP | 3/546 |
| | | G/A(21) | | | | | |
| | | A/A(3) | | | | | |
| ACG→ACA | Thr→Thr | G/G(179) | 565 | Yes | Yes | dbSNP | 3/546 |
| | | G/A(1) | | | | | |
| | | A/A(0) | | | | | |
| ACC→AGC | Thr→Ser | C/C(187) | 626 | Yes | No | dbSNP | 262/546 |
| | | C/G(1) | | | | | |
| | | G/G(0) | | | | | |
| CTG→CTA | Leu→Leu | G/G(187) | 568 | Yes | No | dbSNP | 101/546 |
| | | G/A(1) | | | | | |
| | | A/A(0) | | | | | |
| GGG→GGA | Gly→Gly | G/G(188) | 378 | Yes | Yes | dbSNP | 71/546 |
| | | G/A(1) | | | | | |
| | | A/A(0) | | | | | |
| ACG→ATG | Thr→Met | C/C(187) | 383 | Yes | Yes | HGMD | 7/424 |
| | | C/T(1) | | | | | |
| | | T/T(0) | | | | | |

*Relative to Genbank Accession 4557358
**number of individuals may not be consistent if sequence was unreadable at the specified position

TABLE 6
SNPs Discovered in EDNRA

| | | | | | | | Codon |
|---|---|---|---|---|---|---|---|
| Codon | Amino | Genotype | | | | | mutation |
| mutation | acid | (number | | | SNIDE | Matrix | class rank |
| class | change | individuals) | Position* | Novel? | prediction? | type | in matrix |
| CTG->CTA | Leu-> | G/G(184) | 360 | Yes | No | dbSNP | 101/546 |
| | Leu | G/A(0) | | | | | |
| | | A/A(1) | | | | | |

*Relative to Genbank Accession NM_001957
**Number of individuals may not be consistent if sequence was unreadable at the specified position.

TABLE 7
SNPs DISCOVERED IN ADBR1

| | | | | | | | Codon |
|---|---|---|---|---|---|---|---|
| Codon | Amino | Genotype | | | | | mutation |
| mutation | acid | (number | | | SNIDE | Matrix | class rank |
| class | change | individuals) | Position* | Novel? | prediction? | type | in matrix |
| AGC→GGC | Ser→Gly | A/A(134) | 231 | No | No | dbSNP | 125/546 |
| | | A/G(39) | | | | | |
| | | G/G(8) | | | | | |
| GTG→GTA | Val→Val | G/G(178) | 293 | Yes | No | dbSNP | 82/546 |
| | | G/A(2) | | | | | |
| | | A/A(0) | | | | | |
| AAT→AAC | Asn→Asn | T/T(179) | 312 | Yes | Yes | dbSNP | 27/546 |
| | | T/C(1) | | | | | |
| | | C/C(0) | | | | | |
| GTG→GTA | Val→Val | G/G(179) | 323 | Yes | No | dbSNP | 82/546 |
| | | G/A(2) | | | | | |
| | | A/A(0) | | | | | |
| CTG→TTG | Leu→Leu | C/C(179) | 384 | Yes | Yes | dbSNP | 56/546 |
| | | C/T(1) | | | | | |
| | | T/T(0) | | | | | |
| ACC→GCC | Thr→Ala | A/A(180) | 490 | Yes | No | dbSNP | 105/546 |
| | | A/G(1) | | | | | |
| | | G/G(0) | | | | | |
| TGC→TGT | Cys→Cys | C/C(169) | 626 | Yes | Yes | dbSNP | 37/546 |
| | | C/T(5) | | | | | |
| | | T/T(0) | | | | | |

*Relative to Genbank Accession NM_000684
**Number of individuals may not be consistent if sequence was unreadable at the specified position.

Dilated cardiomyopathy (DCM) defines a group of related disorders characterized by cardiac enlargement and weakening as to educe congestive heart failure. Approximately 80% of cases are idiopathic, that is, have no known source. The HGMD lists twenty five SNPs in five genes that have been shown to be causative of the disorder or one of its subtypes.

It was found that three mutations were predicted by SNIDE using the dbSNP matrix and one novel mutation (Thr→Met, ACG→ATG) was predicted using the pSNP-finding HGMD SNIDE. The putative pSNP occurs in one dilated cardiomyopathy patient in the bradykinin beta receptor 2 gene (BDKRB2), exon 3. This SNP changes a threonine (ACG) at position 128 in the 391-AA protein to a methionine (ATG). BDKRB2 is a G-protein coupled receptor that spans the cell membrane and associates with G-proteins that activate a phosphatidylinositol-calcium second messenger system. Replacing the Thr with a Met may potentially alter the protein structure as to cause a phenotype.

The SNIDE algorithm relies upon aggregate properties of a large mutation dataset, which reflect a likelihood of mutation occurrence and impact, which are used to approximate the local mutational properties of any given gene. It is clear from the data in TABLE 2, however, that the impact portion of the predictiveness number may be modified. For example, a Val→Ile mutation may have little or no impact on a protein in most situations, but if it happens to be in a position important for folding or function then the mutation may be causative of some disease. Therefore, the addition of gene-specific factors regarding impact should increase the accuracy of SNIDE. One method for improving accuracy is to analyze conservative versus non-conservative substitutions under the premise that such crucial residues will be conserved roughly proportional to their importance. Homolog searches, 3D structure comparisons, coupling (mutual information), and secondary structure predictions are all components that may be added into SNIDE to modulate predictions based on projected impact.

Even with an available protein structure, it may be difficult to forecast the effects of a mutation because residues may have interactions with unknown members of biochemical pathways or the mutation may disrupt folding, thereby causing a phenotype, but not alter function in the final folded state. For example, there may be some verified missense mutations, however, that do not occur at a highly conserved residue. The lack of conservation may be because the discovered mutations are not causative of disease, but rather, linked to the true causative allele somewhere else in the gene or gene cluster. Additionally, some verified mutations that are not over-represented in the affected population may increase the predictiveness rank upon positional weighting because the allele does in fact cause disease, but not the disease being studied.

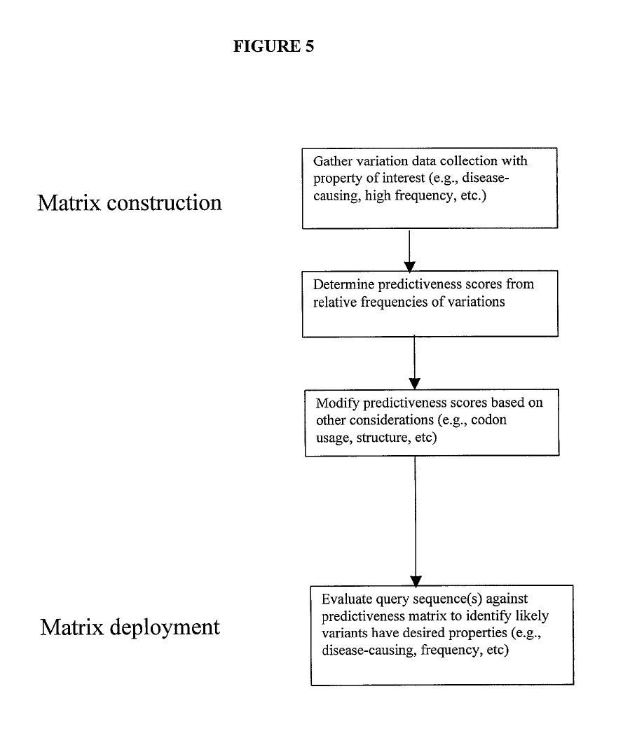

Another way to reclassify predictions by impact is to consider the effects of the mutation on both DNA and mRNA structure. Such mutations may have negligible effect on the resulting protein structure in the final product but disrupt seriously transcription or translation. One scenario is that a mutation may favor the formation of a thermodynamically stable hairpin in unwound single-stranded DNA that causes the RNA polymerase to skip a chunk of sequence and generate a frameshift deletion in the protein. Knowledge of protein structure and amino acid conservation is useful to tailor the mutation predictions even further towards a high impact data set, mRNA and DNA structure may be either predicted (using commercial packages such as MFOLD) or detected experimentally in vitro. FIG. 5 depicts the matrix construction and deployment process when using SNIDE.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

While this invention has been described in reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

List of Identified Alleles (SEQ ID NO.: 1-12)

| | |
|---|---|
| BDKRB2;GI:4557358;position 565 | SEQ ID NO.:1 |

GCTGGGCCAAGCTCTACAGCTTGGTGATCTGGGGGTGTACGCTGCTCCTG
[G/A]GCTCACCCATGCTGGTGTTCCGGACCATGAAGGAGTACAGCGATG
AGGGC

| | |
|---|---|
| BDKRB2;GI:4557358;position 626 | SEQ ID NO.:2 |

GCTGGTGTTCCGGACCATGAAGGAGTACAGCGATGAGGGCCACAACGTCA
[C/G]CGCTTGTGTCATCAGCTACCCATCCCTCATCTGGGAAGTGTTCAC
CAACA

| | |
|---|---|
| BDKRB2;GI:4557358;position 568 | SEQ ID NO.:3 |

GGGCCAAGCTCTACAGCTTGGTGATCTGGGGGTGTACGCTGCTCCTGAGC
[G/A]CACCCATGCTGGTGTTCCGGACCATGAAGGAGTACAGCGATGAG
GGCCAC

| | |
|---|---|
| BDKRB2;GI:4557358;position 378 | SEQ ID NO.:4 |

CTGCCCTTCTGGGCCATCACCATCTCCAACAACTTCGACTGGCTCTTTGG
[G/A]GAGACGCTCTGCCGCGTGGTGAATGCCATTATCTCCATGAACCT
GTACAG

| | |
|---|---|
| BDKLRB2;GI:4557358;position 383 | SEQ ID NO.:5 |

CTTCTGGGCCATCACCATCTCCAACAACTTCGACTGGCTCTTTGGGGAGA
[C/T]GCTCTGCCGCGTGGTGAATGCCATTATCTCCATGAACCTGTACA
GCAGCA

| | |
|---|---|
| EDNRA;GB:NM_001957;position 360 | SEQ ID NO.:6 |

GGACACCGGCCACCCTCCGCGCCACCCACCCTCGCTTTCTCCGGCTTCCT
[G/A]TGGCCCAGGCGCCGCGCGGACCCGGCAGCTGTCTGCGCACGCCG
AGCTCC

| | |
|---|---|
| ADBR1;GB:NM_000684;position 293 | SEQ ID NO.:7 |

CTGTCTCAGCAGTGGACAGCGGGCATGGGTCTGCTGATGGCGCTCATCGT
[G/A]CTGCTCATCGTGGCGGGCAATGTGCTGGTGATCGTGGCCATCGC
CAAGAC

| | |
|---|---|
| ADBR1;GB:NM_000684;position 312 | SEQ ID NO.:8 |

CGGGCATGGGTCTGCTGATGGCGCTCATCGTGCTGCTCATCGTGGCGGGC
[T/C]ATGTGCTGGTGATCGTGGCCATCGCCAAGACGCCGCGGCTGCAG
ACGCTC

| | |
|---|---|
| ADBR1;GB:NM_000684;position 323 | SEQ ID NO.:9 |

CTGCTGATGGCGCTCATCGTGCTGCTCATCGTGGCGGGCAATGTGCTGGT
[G/A]ATCGTGGCCATCGCCAAGACGCCGCGGCTGCAGACGCTCACCAA
CCTCTT

| | |
|---|---|
| ADBR1;GB:NM_000684;position 384 | SEQ ID NO.:10 |

TCGCCAAGACGCCGCGGCTGCAGACGCTCACCAACCTCTTCATCATGTCC
[C/T]TGGCCAGCGCCGACCTGGTCATGGGGCTGCTGGTGGTGCCGTTC
GGGGCC

| | |
|---|---|
| ADBR1;GB:NM_000684;position 490 | SEQ ID NO.:11 |

CGTGGTGTGGGGCCGCTGGGAGTACGGCTCCTTCTTCTGCGAGCTGTGGA
[A/G]CTCAGTGGACGTGCTGTGCGTGACGGCCAGCATCGAGACCCTGT
GTGTCA

| | |
|---|---|
| ADBR1;GB:NM_000684;position 626 | SEQ ID NO.:12 |

TTCCGCTACCAGAGCCTGCTGACGCGCGCGCGGGCGCGGGGCCTCGTGTG
[C/T]ACCGTGTGGGCCATCTCGGCCCTGGTGTCCTTCCTGCCCATCCT
CATGCA

What is claimed is:

1. A method for predicting single nucleotide polymorphisms, comprising the steps of:
obtaining a variation predictiveness matrix; and
predicting one or more single nucleotide polymorphisms of a nucleic acid sequence based on the variation predictiveness matrix.

2. The method of claim 1 further comprising one or more nucleic acid sequences with chemical modifications.

3. The method of claim 2, wherein the chemical modifications include methylation or other chemical groups that incorporate additional charge, polarizability, hydrogen bonding, electrostatic interaction, and fluxionality to the individual nucleic acid bases or to the nucleic acid sequence as a whole.

4. The method of claim 1, wherein the step of predicting the likelihood of one or more single nucleotide polymorphisms comprises the steps of:
comparing the nucleic acid sequence one or more bases at a time with the variation predictiveness matrix to assign a variation value to bases in the nucleic acid sequence; and
selecting the polymorphisms that will likely cause a variation in one or more bases of the nucleic sequence based on the variation value.

5. The method of claim 4, wherein the variation in one or more bases is nonsynonymous.

6. The method of claim 4, wherein the variation in one or more bases is synonymous.

7. The method of claim 1, further comprising the step of generating a dataset of single nucleotide polymorphisms for one or more nucleic acid sequences.

8. The method of claim 1, wherein the step of obtaining a variation predictiveness matrix, further comprises the steps of:
calculating a variation frequency from a first base to a second base in a dataset of two or more genes; and
generating the variation predictiveness matrix from the calculated variation frequency.

9. The method of claim 8 wherein the dataset comprises genes with nucleic acid chemical modifications.

10. The method of claim 9, wherein the chemical modifications include methylation or other chemical groups that incorporate additional charge, polarizability, hydrogen bonding, electrostatic interaction, and fluxionality to the individual nucleic acid bases or to the nucleic acid as a whole.

11. The method of claim 8, wherein the variation frequency is determined from a known mutation dataset.

12. The method of claim 8, wherein the variation frequency is determined from a dataset of known diseases.

13. The method of claim 8, wherein the variation frequency is determined from a dbSNP database.

14. The method of claim 8, wherein the variation frequency is determined from a non-human mutation database.

15. The method of claim 8, wherein the variation frequency is determined from a disease-specific database.

16. The method of claim 8, wherein the variation frequency is determined from a non-human disease database.

17. The method of claim 8, wherein the variation frequency is determined from a HGMD database.

18. The method of claim 8, wherein the variation frequency is determined from a linkage database.

19. The method of claim 8, wherein the variation frequency is determined from a splice variant database.

20. The method of claim 8, wherein the variation frequency is determined from a translocation database.

21. The method of claim 8, wherein the variation frequency is determined from a database of known mutations.

22. The method of claim 8, wherein the variation frequency is further adjusted for wild type genes.

23. The method of claim 8, wherein the variation frequency is further adjusted for engineered or non-naturally occurring genes.

24. The method of claim 8, wherein the variation frequency is further adjusted for conservative polymorphisms.

25. The method of claim 8, wherein the variation frequency is further adjusted for non-conservative polymorphisms.

26. The method of claim 8, wherein the variation frequency is further adjusted for cDNA stability.

27. The method of claim 8, wherein the variation frequency is further adjusted for predicted DNA structure.

28. The method of claim 8, wherein the variation frequency is further adjusted for predicted RNA structure.

29. The method of claim 8, wherein the variation frequency is further adjusted for predicted protein structure.

30. The method of claim 8, wherein the variation frequency is further adjusted for post-translational modification sequences.

31. The method of claim 8, wherein the variation frequency is further adjusted for protein stability.

32. The method of claim 8, wherein the variation frequency is further adjusted for predicted protein transport.

33. The method of claim 8, wherein the variation frequency is further adjusted for shuffled genes.

34. The method of claim 8, wherein the variation frequency is further adjusted for site-directed mutagenesis genes.

35. The method of claim 8, wherein the variation frequency is further adjusted for methylated sequences

36. The method of claim 8, wherein the variation frequency is further adjusted for epigenetic variation.

37. The method of claim 8, wherein the nucleic acid sequence comprises a cDNA sequence.

38. The method of claim 8, wherein the nucleic acid sequence comprises genomic sequence.

39. The method of claim 8, wherein the nucleic acid sequence comprises an intron/exon boundary.

40. The method of claim 8, wherein the nucleic acid sequence comprises a transcriptional control sequence.

41. The method of claim 8, wherein the nucleic acid sequence comprises a transport control sequence.

42. The method of claim 8, wherein the nucleic acid sequence comprises a translational control sequence.

43. The method of claim 8, wherein the nucleic acid sequence comprises a transcriptional control sequence.

44. The method of claim 8, wherein the nucleic acid sequence comprises a splicing control sequence.

45. The method of claim 1, wherein the step of obtaining a variation predictiveness matrix correlates the frequency of a first codon mutation to a second codon mutation with a variation predictiveness value of a nucleic acid sequence from one to ten bases at a time.

46. The method of claim 1, wherein in the variation predictiveness matrix is normalized for the codon usage of a target organism.

47. The method of claim 1, wherein the variation predictiveness matrix is generated from a mutant gene dataset that comprises all mutant genes in a mutant gene database.

48. The method of claim 1, wherein the variation predictiveness matrix is generated from a mutant gene dataset that comprises all mutant genes in a mutant gene database minus the known mutant genes of the mutant gene dataset.

49. The method of claim 1, where the nucleic acid sequence comprises an entire genome.

50. The method of claim 1, where the nucleic acid sequence comprises a human genome.

51. The method of claim 1, where the nucleic acid sequence comprises a gene cluster for a target human disease.

52. The method of claim 1, where the variation predictiveness matrix is based on a mutant gene dataset that comprises a human mutation database.

53. The method of claim 1, wherein the steps are affected by a computer program.

54. The method of claim 53, wherein the computer program is SNIDE.

55. The method of claim 53, wherein the computer program is SNooP.

56. The method of claim 1, wherein the variation predictiveness matrix is determined in silico from a human mutant database.

57. The method of claim 1, wherein the step of predicting a likelihood of one or more single nucleotide polymorphisms is determined in silico.

58. A method for creating a variation predictiveness value for use in a variation predictiveness matrix, comprising the steps of:
calculating the variation frequency from a first nucleic acid to a second nucleic acid in a dataset of two or more variations; and
determining a variation predictiveness value from the calculated variation frequency.

59. The method of claim 58, further comprising the step of generating a variation predictiveness matrix that correlates the frequency of a first to a second variation with the variation predictiveness value.

60. The method of claim 58, wherein the dataset comprises genes with nucleic acid chemical modifications.

61. The method of claim 60, wherein the chemical modifications include methylation or other chemical groups that incorporate additional charge, polarizability, hydrogen bonding, electrostatic interaction, and fluxionality to the individual nucleic acid bases or to the nucleic acid as a whole.

62. The method of claim 58, wherein the variation frequency is determined from a known mutation dataset.

63. The method of claim 58, wherein the variation frequency is determined from a dataset of known diseases.

64. The method of claim 58, wherein the variation frequency is determined from a dbSNP database.

65. The method of claim 58, wherein the variation frequency is determined from a non-human mutation database.

66. The method of claim 58, wherein the variation frequency is determined from a disease-specific database.

67. The method of claim 58, wherein the variation frequency is determined from a non-human disease database.

68. The method of claim 58, wherein the variation frequency is determined from a HGMD database.

69. The method of claim 58, wherein the variation frequency is determined from a linkage database.

70. The method of claim 58, wherein the variation frequency is determined from a splice variant database.

71. The method of claim 58, wherein the variation frequency is determined from a translocation database.

72. The method of claim 58, wherein the variation frequency is determined from a database of known mutations.

73. The method of claim 58, wherein the variation frequency is further adjusted for wild type genes.

74. The method of claim 58, wherein the variation frequency is further adjusted for engineered or non-naturally occurring genes.

75. The method of claim 58, wherein the variation frequency is further adjusted for conservative polymorphisms.

76. The method of claim 58, wherein the variation frequency is further adjusted for non-conservative polymorphisms.

77. The method of claim 58, wherein the variation frequency is further adjusted for cDNA stability.

78. The method of claim 58, wherein the variation frequency is further adjusted for predicted DNA structure.

79. The method of claim 58, wherein the variation frequency is further adjusted for predicted RNA structure.

80. The method of claim 58, wherein the variation frequency is further adjusted for predicted protein structure.

81. The method of claim 58, wherein the variation frequency is further adjusted for post-translational modification sequences.

82. The method of claim 58, wherein the variation frequency is further adjusted for protein stability.

83. The method of claim 58, wherein the variation frequency is further adjusted for predicted protein transport.

84. The method of claim 58, wherein the variation frequency is further adjusted for shuffled genes.

85. The method of claim 58, wherein the variation frequency is further adjusted for site-directed mutagenesis genes.

86. The method of claim 58, wherein the variation frequency is further adjusted for methylated sequences

87. The method of claim 58, wherein the variation frequency is further adjusted for epigenetic variation.

88. The method of claim 58, wherein the variations comprise a cDNA sequence.

89. The method of claim 58, wherein the variations comprise genomic sequence.

90. The method of claim 58, wherein variations comprise an intron/exon boundary.

91. The method of claim 58, wherein variations comprise exons.

92. The method of claim 58, wherein variations comprise other SNPs.

93. The method of claim 58, wherein variations comprise inversions.

94. The method of claim 58, wherein variations comprise deletions.

95. The method of claim 58, wherein variations comprise splice variations.

96. The method of claim 58, wherein variations comprise translocations.

97. The method of claim 58, wherein variations comprise a transcriptional control sequence.

98. The method of claim 58, wherein variations comprise a transport control sequence.

99. The method of claim 58, wherein variations comprise a translational control sequence.

100. The method of claim 58, wherein variations comprise a transcriptional control sequence.

101. The method of claim 58, wherein variations comprise a splicing control sequence.

102. The method of claim 59, wherein in the variation predictiveness matrix is normalized for the nucleotide usage of a target organism.

103. The method of claim 59, wherein the variation predictiveness matrix is generated from a mutant gene dataset that comprises all mutant genes in a mutant gene database.

104. The method of claim 58, wherein the variation predictiveness matrix is generated from a mutant gene dataset that comprises all mutant genes in a mutant gene database minus the known mutant genes of the mutant gene dataset.

105. The method of claim 58, where the nucleic acid comprises one or more bases.

106. The method of claim 58, where the nucleic acid comprises DNA.

107. The method of claim 58, where the nucleic acid comprises RNA.

108. The method of claim 58, where the nucleic acid comprises a triplet.

109. The method of claim 58, The method of claim 16, where the nucleic acid comprises a codon.

110. The method of claim 58, The method of claim 16, where the nucleic acid comprises one or more non-sequence base modifications.

111. The method of claim 58, where the nucleic acid comprises modified nucleic acids.

112. The method of claim 58, wherein modified nucleic acids include methylation or other chemical groups that incorporate additional charge, polarizability, hydrogen bonding, electrostatic interaction, and fluxionality to the individual nucleic acid bases or to the nucleic acid as a whole.

113. The method of claim 58, where the nucleic acid comprises an entire genome.

114. The method of claim 58, where the nucleic acid comprises a human genome.

115. The method of claim 58, where the nucleic acid comprises a gene cluster for a target human disease.

116. The method of claim 58, where the variation predictiveness matrix is based on a mutant gene dataset that comprises a human mutation database.

117. The method of claim 58, wherein the steps are affected by a computer program.

118. The method of claim 58, wherein the computer program is SNIDE.

119. The method of claim 58, wherein the computer program is SNooP.

120. The method of claim 58, wherein the variation predictiveness value is determined in silico from a human mutant database.

121. The method of claim 58, wherein the step of predicting a likelihood of one or more single nucleotide variation is determined in silico.

122. A method for creating a polymorphism predictiveness value for use in a mutation predictiveness matrix, comprising the steps of:
calculating the mutation frequency from a first codon to a second codon in a dataset of two or more mutant genes; and
determining a polymorphism predictiveness value from the calculated mutation frequency.

123. The method of claim 122, further comprising the step of generating a codon polymorphism predictiveness matrix that correlates the frequency of a first to a second codon mutation with the polymorphism predictiveness value.

124 The method of claim 122, wherein the dataset comprises nucleic acids with chemical modifications.

125 The method of claim 124, wherein the chemical modifications include methylation or other chemical groups that incorporate additional charge, polarizability, hydrogen bonding, electrostatic interaction, and fluxionality to the individual nucleic acid bases or to the nucleic acid as a whole.

126 The method of claim 122, wherein the mutation frequency is determined from a known mutation dataset.

127 The method of claim 122, wherein the mutation frequency is determined from a dataset of known diseases.

128 The method of claim 122, wherein the mutation frequency is determined from a dbSNP database.

129 The method of claim 122, wherein the mutation frequency is determined from a non-human mutation database.

130 The method of claim 122, wherein the mutation frequency is determined from a disease-specific database.

131 The method of claim 122, wherein the mutation frequency is determined from a non-human disease database.

132. The method of claim 122, wherein the mutation frequency is determined from a HGMD database.

133. The method of claim 122, wherein the mutation frequency is determined from a linkage database.

134. The method of claim 122, wherein the mutation frequency is determined from a splice variant database.

135. The method of claim 122, wherein the mutation frequency is determined from a translocation database.

136. The method of claim 122, wherein the mutation frequency is determined from a database of known mutations.

137. The method of claim 122, wherein the mutation frequency is further adjusted for wild type genes.

138. The method of claim 122, wherein the mutation frequency is further adjusted for engineered or non-naturally occurring genes.

139. The method of claim 122, wherein the mutation frequency is further adjusted for conservative polymorphisms.

140. The method of claim 122, wherein the mutation frequency is further adjusted for non-conservative polymorphisms.

141. The method of claim 122, wherein the mutation frequency is further adjusted for cDNA stability.

142. The method of claim 122, wherein the mutation frequency is further adjusted for predicted DNA structure.

143. The method of claim 122, wherein the mutation frequency is further adjusted for predicted RNA structure.

144. The method of claim 122, wherein the mutation frequency is further adjusted for predicted protein structure.

145. The method of claim 122, wherein the mutation frequency is further adjusted for post-translational modification sequences.

146. The method of claim 122, wherein the mutation frequency is further adjusted for protein stability.

147. The method of claim 122, wherein the mutation frequency is further adjusted for predicted protein transport.

148. The method of claim 122, wherein the mutation frequency is further adjusted for shuffled genes.

149. The method of claim 122, wherein the mutation frequency is further adjusted for site-directed mutagenesis genes.

150. The method of claim 122, wherein the mutation frequency is further adjusted for methylated sequences

151. The method of claim 122, wherein the mutation frequency is further adjusted for epigenetic variation.

152. The method of claim 122, wherein the mutant genes comprise a cDNA sequence.

153. The method of claim 122, wherein the mutant genes comprise genomic sequence.

154. The method of claim 122, wherein mutant genes comprise an intron/exon boundary.

155. The method of claim 122, wherein mutant genes comprise exons.

156. The method of claim 122, wherein mutant genes comprise other SNPs.

157. The method of claim 122, wherein mutant genes comprise inversions.

158. The method of claim 122, wherein mutant genes comprise deletions.

159. The method of claim 122, wherein mutant genes comprise splice variations.

160. The method of claim 122, wherein mutant genes comprise translocations.

161. The method of claim 122, wherein mutant genes comprise a transcriptional control sequence.

162. The method of claim 122, wherein mutant genes comprise a transport control sequence.

163. The method of claim 122, wherein mutant genes comprise a translational control sequence.

164. The method of claim 122, wherein mutant genes comprise a transcriptional control sequence.

165. The method of claim 122, wherein mutant genes comprise a splicing control sequence.

166. The method of claim 123, wherein in the codon polymorphism predictiveness matrix is normalized for the codon usage of a target organism.

167. The method of claim 123, wherein the codon polymorphism predictiveness matrix is generated from a mutant gene dataset that comprises all mutant genes in a mutant gene database.

168. The method of claim 123, wherein the codon polymorphism predictiveness matrix is generated from a mutant gene dataset that comprises all mutant genes in a mutant gene database minus the known mutant genes of the mutant gene dataset.

169. The method of claim 122, where the codon comprises one or more bases.

170. The method of claim 122, where the codon comprises DNA.

171. The method of claim 122, where the codon comprises RNA.

172. The method of claim 122, where the codon comprises a triplet.

173. The method of claim 122, where the codon comprises a codon.

174. The method of claim 122, where the codon comprises one or more non-sequence base modifications.

175. The method of claim 122, wherein the codon further comprises modifications.

176. The method of claim 122, wherein modifications include methylation or other chemical groups that incorporate additional charge, polarizability, hydrogen bonding, electrostatic interaction, and fluxionality to the individual nucleic acid bases or to the nucleic acid as a whole.

177. The method of claim 122, where the codon comprises an entire genome.

178. The method of claim 122, where the codon comprises a human genome.

179. The method of claim 122, where the codon comprises a gene cluster for a target human disease.

180. The method of claim 122, where the codon polymorphism predictiveness matrix is based on a mutant gene dataset that comprises a human mutation database.

181. The method of claim 122, wherein the step of predicting a likelihood of one or more single nucleotide polymorphisms is determined in silico.

182. A method for creating a variation predictiveness matrix, comprising the steps of:
calculating the variation frequency from a first nucleic acid to a second nucleic acid in a dataset of two or more variations;
determining a variation predictiveness value from the calculated variation frequency; and
generating a variation predictiveness matrix that correlates the frequency of a first to a second nucleic acid with the variation predictiveness value.

183. The method of claim 182, wherein the dataset comprises nucleic acids with chemical modifications.

184. The method of claim 183, wherein the chemical modifications include methylation or other chemical groups that incorporate additional charge, polarizability, hydrogen bonding, electrostatic interaction, and fluxionality to the individual nucleic acid bases or to the nucleic acid as a whole.

185. The method of claim 182, wherein the variation frequency is determined from a variation dataset.

186. A method for creating a polymorphism predictiveness matrix, comprising the steps of:
calculating the mutation frequency from a first codon to a second codon in a dataset of two or more mutant genes;
determining a polymorphism predictiveness value from the calculated mutation frequency; and
generating a codon polymorphism predictiveness matrix that correlates the frequency of a first to a second codon mutation with the polymorphism predictiveness value.

187. The method of claim 186, wherein the dataset comprises nucleic acids with chemical modifications.

188. The method of claim 187, wherein the chemical modifications include methylation or other chemical groups that incorporate additional charge, polarizability, hydrogen bonding, electrostatic interaction, and fluxionality to the individual nucleic acid bases or to the nucleic acid as a whole.

189. The method of claim 186, wherein in the codon polymorphism predictiveness matrix is normalized for the codon usage of a target organism.

190. The method of claim 186, wherein the codon polymorphism predictiveness matrix is generated from a mutant gene dataset that comprises all mutant genes in a mutant gene database.

191. The method of claim 186, wherein the codon polymorphism predictiveness matrix is generated from a mutant gene dataset that comprises all mutant genes in a mutant gene database minus the known mutant genes of the mutant gene dataset.

192. The method of claim 186, wherein the codon comprises one or more bases.

193. The method of claim 186, where the codon comprises a triplet.

194. The method of claim 186, where the codon comprises a codon.

195. The method of claim 186, where the codon comprises one or more non-sequence base modifications.

196. An isolated and purified nucleic acid comprising a predicted single nucleotide variation of a nucleic acid sequence based on the variation predictiveness matrix sequence of claim 1.

197. An isolated and purified nucleic acid comprising a predicted single nucleotide polymorphism of a wild-type gene sequence based on the codon mutation predictiveness matrix sequence of claim 1.

198. An apparatus for detecting a single nucleotide polymorphism comprising:
a substrate; and
one or more isolated and purified nucleic acids comprising a predicted single nucleotide variation of a nucleic acid sequence based on a variation predictiveness matrix sequence affixed to the substrate.

199. The apparatus of claim 198, wherein the substrate comprises a microfabricated solid surface to which molecules may be attached through either covalent or non-covalent bonds.

200. The apparatus of claim 198, wherein the substrate further comprises Langmuir-Bodgett films, glass, functionalized glass, germanium, silicon, PTFE, polystyrene, gallium arsenide, gold, silver, or any materials comprising amino, carboxyl, thiol or hydroxyl functional groups incorporated on a planar or spherical surface.

201. An apparatus for detecting a single nucleotide polymorphism comprising:
a substrate; and
one or more isolated and purified nucleic acids comprising a predicted single nucleotide polymorphism of a wild-type gene sequence based on a codon polymorphism predictiveness matrix. sequence affixed to the substrate.

202. The apparatus of claim 201, wherein the substrate comprises a microfabricated solid surface to which molecules may be attached through either covalent or non-covalent bonds.

203. A computer program embodied on a computer readable medium for predicting variations, comprising:
a code segment for creating variation predictiveness matrix from a nucleic acid dataset;
a code segment for comparing a wild-type gene sequence with the variation predictiveness matrix; and
a code segment for predicting variations in the wild-type gene sequence based on the comparison.

204. A computer program embodied on a computer readable medium for predicting polymorphisms, comprising:
a code segment for creating a codon mutation predictiveness matrix from a mutant gene dataset;
a code segment for comparing a wild-type gene sequence with the codon polymorphism predictiveness matrix; and
a code segment for predicting polymorphisms in the wild-type gene sequence based on the comparison.

205. A polymorphism prediction dataset, comprising:
a first nucleic acid;
a second nucleic acid variation that correlates to a polymorphism from the first nucleic acid; and
a variation predictiveness value determined from known variations in a variation database for a target organism.

206. A polymorphism prediction dataset, comprising:
a first codon;
a second codon mutation that correlates to a mutation from the first codon; and
a codon polymorphism predictiveness value determined from known mutations in a mutation database for a target organism.

207. A single nucleotide polymorphism determined by the method of claim 1.

208. A method for predicting single nucleotide polymorphisms, comprising the steps of:
inputing each codon in a queried nucleic acid sequence;
determining each possible nonsynonymous mutation;
assigning a predictiveness value to that mutation based on the identity of the wild-type and resultant codon; and
ranking of all predictiveness values to highlight the likely to occur and impact gene function.

209. The method of claim 208, further comprising the steps of:
parsing one or more nucleic acid sequence input files having sequence information;
calculating an expected mutation liklihood according to a user-defined threshold; and
ranking of point mutation predictions by a ζ-value.

210. The method of claim 208, further comprising the step of generating a delimited file suitable for a standard spreadsheet application.

211. An isolated and purified nucleic acid comprising SEQ ID NOS.: 1-12.

212. An isolated and purified nucleic acid comprising a cardiomyopathy disease related SNP selected from the group consisting essentially of BDKRB2, EDNRA, ADRB1, ADRB2, CREB1 and MCIP.

213. An isolated and purified nucleic acid of claim 211, wherein the SNP is Thr→Met substitution in BDKRB2 at position 383.
